(19) United States
(12) Patent Application Publication
(10) Pub. No.: US 2012/0039888 A1
(43) Pub. Date: Feb. 16, 2012

(54) Methods Of Inhibiting Inflammation-Associated Tissue Damage By Inhibiting Neutrophil Activity

(76) Inventors: Paul Frenette, New York, NY (US); Andrés Hidalgo, Madrid (ES)

(21) Appl. No.: 13/104,798

(22) Filed: May 10, 2011

Related U.S. Application Data
(63) Continuation of application No. PCT/US2009/063916, filed on Nov. 10, 2009.
(60) Provisional application No. 61/113,112, filed on Nov. 10, 2008.

(51) Int. Cl. A61K 39/395; A61K 31/404; A61K 31/506; A61K 31/4365; A61K 31/519; A61K 38/44; A61K 31/375; A61K 31/355; A61K 31/522; A61K 38/06; A61K 31/197; A61K 31/03; A61P 29/00; A61P 11/00; A61K 31/713

(52) U.S. Cl. 424/138.1; 424/172.1; 514/44 A; 514/414; 514/252.19; 514/301; 514/265.1; 514/262.1; 424/94.4; 514/474; 514/458; 514/263.33; 514/12.2; 514/562; 514/753

(57) ABSTRACT

The present invention relates to compositions and methods for treating inflammation-associated tissue damage, and particularly transfusion-associated, disorders, by inhibiting or reducing E-selectin mediated signal transduction pathways, such as E-selectin mediated activation of αMβ2.

### CROSS-REFERENCE TO RELATED APPLICATION

The present application is a continuation of International Patent Application No. PCT/US09/063916, filed Nov. 10, 2009, which claims the benefit of U.S. Provisional Application No. 61/113,112, filed Nov. 10, 2008, both of which are hereby incorporated by reference in their entireties.

### GRANT INFORMATION

The subject matter of this invention was developed, at least in part, under National Institutes of Health Grants RO1 HL69438 and T32 HL07824, so that the United States Government holds certain rights herein.

### 1. INTRODUCTION

The present invention relates to compositions and methods for treating inflammatory, and particularly transfusion-associated, disorders.

### 2. BACKGROUND OF THE INVENTION

Transfusion-related acute lung injury (TRALI), the leading cause of transfusion-related morbidity and mortality, is an acute inflammatory process mediated by intravascular neutrophils. Neutrophil and platelet recruitment and activation play key roles in the acute inflammatory response. Leukocyte recruitment is initiated by interactions with endothelial selectins, followed by activation of integrins which interact with their counter-receptors on the inflamed endothelium. Before migrating into the surrounding tissue, neutrophils actively crawl on the inflamed endothelium displaying a characteristic polarization with distinct microdomains at the leading (LE) and trailing edges (TE). During this period, they establish frequent interactions with circulating erythrocytes through the LE, which in the context of sickle cell disease can lead to vascular occlusion.

### 3. SUMMARY OF THE INVENTION

The present invention relates to compositions and methods for inhibiting or reducing inflammation-associated tissue damage, and particularly tissue and organ damage in the context of a transfusion reaction (especially Transfusion-Related Acute Lung Injury or “TRALI”). It is based, at least in part, on the discovery that, in the context of inflammation and/or an animal model of TRALI, tissue/organ damage is effected through E-selectin-mediated activation of Mac-1 (via ESL-1 and Src family members such as Src, Yes, Fyn, Fgr, Lck, Hck, Blk, Lyn and Frk) and subsequent generation of reactive oxygen species (“ROS”). According to the present invention, inhibition of any of these steps may be used to inhibit tissue/organ damage.

### 4. BRIEF DESCRIPTION OF THE DRAWINGS

FIG. 1a-e shows heterotypic interactions of RBC and platelets with leukocyte microdomains are induced during inflammation. (a) Heterotypic interactions between leukocytes and platelets in a TNF-α-stimulated C57BL/6 mouse. Intravenous injection of a fluorescently conjugated antibody to L-selectin (blue) and CD41 (red) allows the identification of the trailing edge of crawling leukocytes and platelets, respectively. The short arrow indicates an interacting RBC, and the asterisks show interacting platelets (green for those mediated by the trailing edge, white for the leading edge). The large arrow points to the direction of blood flow. Scale bar=10 μm. (b) Frequency of interactions between RBCs and leukocytes in venules of mice where surgical trauma or trauma plus TNF-α administration preceded imaging. n=5 mice. ***, p<0.001, Mann-Whitney test. (c) Frequency of interactions between leukocytes and platelets. n=5-6 mice. ***, p<0.001, Mann-Whitney test. (d) Contribution of the leading and trailing edges in RBC interactions after TNF-α treatment. n=4 mice; ‡, p<0.005. (e) Contribution of leukocyte microdomains to platelet interactions in wild-type mice, treated or not with TNF-α. n=5 mice per group; I, p<0.005 compared to Trailing group, paired t-test; n.s., not significant. Bars represent mean±SEM.

FIG. 2a-f shows red blood cell (RBC) and platelet interactions depend on E-selectin and its ligand ESL-1. (a) The number of RBC captures per adherent leukocyte is reduced in Sele^{-/-} mice compared to wild-type controls or Selp^{-/-} animals. n=32-40 venules from 5-7 mice per group. *, p<0.05 compared to WT, **, p<0.01 compared to the other groups; Kruskal-Wallis test with Dunn's multigroup comparison. (b) Reduced number of platelet captures per adherent leukocyte in Sele^{-/-} mice. n=32-42 venules from at least 5 mice per group. ***, p<0.0001. (c) Contribution of leukocyte microdomains to platelet interactions in TNF-α-treated mice. n=5 mice per group. **, p<0.01, Mann-Whitney test. ‡, p=0.003 and n.s., not significant compared to Trailing groups, paired t-test. (d) RBC-leukocyte interactions in wild-type, Selplg^{-/-} and Cd44^{-/-} mice. n=31-42 venules from 5-7 mice per group. *, p<0.05, Kruskal-Wallis test with Dunn's multigroup comparison. (e) RBC captures by leukocytes require the expression of ESL-1 as determined in mouse chimera harboring leukocytes transduced with control (Scrmbl) or shESL-1 lentiviral vectors. The frequency of interactions was determined and expressed as ratio of transduced (GFPP^{pos}) and non-transduced (GFP^{neg}) leukocytes adhered in the same venular segments. Bars represent mean±SEM. n=4-6 mice per group. ***, p<0.0001 compared to GFP^{neg} cells from the same lentiviral group, paired t-test. Bars represent mean±SEM. (f) Inhibition of nRBC (normal red blood cell) interactions in mice pretreated with a Src kinase inhibitor (PP2), but not with p38 MAPK (SB203580) or Syk (Piceatannol) inhibitors, compared to vehicle control (DMSO). n=33-44 venules from 6 mice per group. *, p<0.05 compared to all other groups, Kruskal-Wallis test with Dunn's multigroup comparison. Bars represent mean±SEM.

FIG. 3a-d shows that heterotypic interactions with RBC and platelets are mediated by the leukocyte integrin αMβ2. (a) Micrographs of adherent leukocytes obtained by MFIM analyses of TNF-α-treated C57BL/6 mice injected with 0.5 μg of PE-conjugated anti-L-selectin (red) and 1 μg of APC-conjugated anti-αM (green) Abs. αMβ2 integrin expression is homogenously distributed, including where an RBC (arrowhead) is shown interacting at the leading edge of an adherent leukocyte. Scale bar=10 μm. (b) RBC-WBC interactions in Itgam^{-/-} mice compared to wild-type (WT) controls. n=41-42 venules from 6-7 mice per group. **, p<0.001, Mann-Whitney test. (c) Platelet-WBC interactions in Itgam^{-/-} mice compared to WT. n=35-36 venules from 5-6 mice per group. **, p<0.0001, Mann-Whitney test. (d) Contribution of leukocyte microdomains to platelet captures in TNF-α-treated Itgam^{-/-} mice. n=5 mice. ‡, p=0.008, paired t-test compared to Trailing values. Bars represent mean±SEM.

FIG. 4a-f shows E-selectin and ESL-1 modulate regional αMβ2 activity on adherent leukocytes in vivo. Albumin-coated fluorospheres were injected into TNF-α-treated mice prepared for intravital microscopy analysis to assess αMβ2 activity on adherent leukocytes. (a) Injection of fluorescently conjugated antibodies to Gr-1 and F4/80 identifies Gr-1^{pos} F4/80^{neg} PMNs as the subset mediating fluosphere capture. Data obtained from analyses of 84 fluosphere captures from 4 mice. ***, p<0.001, t-test. (b) Time-lapse micrographs of in vivo fluosphere capture by two representative adherent leukocytes (asterisks). For clarity, the leading edge of these leukocytes has been outlined with dotted lines. The first fluosphere is captured at t=10 s (arrowhead) by the leading edge of one leukocyte, away from the L-selectin cluster (red), and then a second leukocyte captures two fluospheres at t=60 s and t=90 s (arrowheads). The histogram (right panel) shows the quantitation of the fluospheres captured by the leading and trailing edges from 98 events identified in 7 mice; ‡, p=0.0005. Scale bar=10 gm. (c) Binding of albumin-coated fluospheres to leukocytes in wild-type and Itgam^{-/-} mice. Each dot represents the average number of fluospheres bound per leukocyte in individual venules. n=40 venules from 4-5 mice per group. **, p<0.001, Mann-Whitney test. (d) Binding of albumin-coated fluospheres to leukocytes in wild-type, Selp^{-/-} and Sele^{-/-} mice. n=33-43 venules from 4-7 mice per group. *, p<0.05 compared to the other groups; Kruskal-Wallis test with Dunn's multigroup comparison. (e) Binding of fluospheres was not altered in Selplg^{-/-} and Cd44^{-/-} mice compared to wild-type controls. n=30-47 venules from 4-6 mice per group. (f) Fluosphere capture is reduced in leukocytes transduced with shESL-1 compared with scrambled sequence lentiviral vectors. n=5 mice per group. ***, p<0.0001 compared to GFP^{neg} cells from the same lentiviral group, paired t-test. All bars represent mean±SEM.

FIG. 5a-k shows that antibody-induced lung injury requires platelet-leukocyte interactions and is blocked by antibodies to E-selectin and αMβ2. (a) Protein accumulation in BAL fluids of Balb/c mice 2h after i.v. administration of 3.5 mg/kg of anti-H2d or control anti-H2b antibodies. Some mice were previously depleted of circulating platelets by i.p. injection of an anti-platelet serum (αPlt); n=7-9 mice. **, p<0.01 one-way ANOVA with Tukey's multigroup comparison. (b) Platelet counts in the blood of mice analyzed in (a). n=9-10 mice; ***, p<0.001 between any two groups. (c) Representative micrographs of platelet-leukocyte interactions in cremasteric venules of TNF-α-treated mice, before (pre-H2d) and after (post-H2d) administration of anti-H2d antibody. Anti-L-selectin (blue) labels the trailing edge and anti-CD41 (red) labels platelets. Arrow indicates the direction of flow. (d) Frequency of platelet-leukocyte interactions before or after anti-H2d treatment in mice pre-treated with control or anti-E-selectin antibodies. §, p<0.01 and n.s., not significant, compared to pre-H2d values, paired t-test analysis. #, p<0.05, unpaired t-test. (e) Relative contribution of leukocyte microdomains to interactions in the rIgG group before and after administration of the anti-H2d antibody. n=5 mice; *, p=0.01. (f) Representative micrographs depicting vascular permeability induced by anti-H2d or control anti-H2b treatment. FITC-dextran (1 mg) was administered i.v. to determine vascular leakage. Scale bar=100 μm. (g) Quantification of FITC-dextran extravasation as a measure of vascular permeability after control anti-H2b or anti-H2d administration in mice previously treated with rat IgG, anti-E-selectin (9A9) or anti-αMβ2 (M1/70) antibodies. n=20-32 venular fields analyzed per group. **, p<0.01; ***, p<0.001; Kruskal-Wallis test with Dunn's multigroup comparison. (h) Quantification of FITC-dextran extravasation in mice pre-treated with preimmune (Ctrl) or anti-platelet serum (αPlt) before anti-H2d administration. n=16-20 venular fields analyzed per group. §, p=0.002, Mann-Whitney test. (i) Effect of P-selectin, E-selectin and αMβ2 inhibition in lung injury. Mice were injected i.v. with control rat IgG or antibodies to P-selectin (RB40), E-selectin (9A9) or anti-αMβ2 (M1/70) followed by anti-H2d or control anti-H2b. Protein content in BAL fluids was measured 2 h later. n=7-13 mice per group. *, p<0.05; **, p<0.001 using one-way ANOVA with Tukey's multigroup comparison test. (j) Percentage of ROS-positive adherent leukocytes before (pre-H2d) and after (post-H2d) H2d infusion in control mice (Ctrl), mice depleted of platelets (αPlt) or treated with an anti-E-selectin antibody (9A9); n=30-53 venules from 4-5 mice per group. *, p<0.05; ***, p<0.001 compared to all other groups, Kruskal-Wallis test with Dunn's multigroup comparison test. (k) Protein content in BAL fluids of mice treated with saline (Ctrl) or n-acetyl-cysteine (N-AcC). n=5-14 mice per group. *, p<0.05, unpaired t-test. All bars represent mean±SEM.

FIG. 6a-g shows that vaso-occlusion in sickle cell disease is induced by sickle RBC-leukocyte interactions that require E-selectin-mediated activation of αMβ2. (a) Individual contributions of P- and E-selectins in sRBC (sickle red blood cell) capture by adherent leukocytes using genetically deficient mice or function-blocking antibodies. E-selectin expression is required for sRBC capture. n=9-17 mice per group. §, p<0.01 compared to time 0-90 in the same group; *, p<0.05; **, p<0.01; ***, p<0.01 compared to the SCD group at the same time point; ¶, p<0.05 compared to the SCD Selp^{-/-} group at the same time point; Kruskal-Wallis test with Dunn's multigroup correction. Values shown are mean±SEM. (b) Analysis of blood flow rates from the experiments shown in (a), at the 181-270 time point. SS, sickle cell; n=9-16 mice; *, p<0.01, t-test with Tukey's multigroup comparison. (c) Representative micrographs showing albumin-coated fluospheres bound to adherent leukocytes in hemizygous (SA) or homozygous (SS) SCD mice. Note the presence of a leukocyte subset that captures multiple beads in SS but not SA mice. Scale bar=10 μm. (d) Binding of albumin-coated fluospheres to leukocytes in SA and SS chimeras with wild-type endothelium or Sele^{-/-} endothelium. Each circle represents the average number of fluospheres bound per leukocyte for an individual venule, and bars represent mean values. n=24-34 venules from 3-5 mice per group. **, p<0.001 compared to the two other groups; Kruskal-Wallis test with Dunn's multigroup comparison. (e) Experimental scheme to assess the role of αMβ2 in vaso-occlusion. (f) αMβ2 blockade reduced the number of sRBC interactions with leukocytes. n=6 mice per group; *, p=0.001. (g) Kaplan-Meier survival curves after TNF-α treatment for isotype control and M1/70 treated groups. n=6 mice per group. Log-rank test, p<0.002. All bars represent mean±SEM.

FIG. 7a-b shows elevated expression of αMβ2 on Selplg−/− leukocytes. Circulating leukocytes from wild-type, Selplg−/− and Cd44−/− mice were stained for αM (Mac-1), L-selectin and αL (LFA-1) expression after RBC lysis. Staining with APC-conjugated anti-αM (clone M1/70), FITC-conjugated anti-αL (clone M17/4) and PE-conjugated anti-L-selectin (clone MEL-14; all from BD Biosciences) was analyzed on the neutrophil population gated on the basis of side and forward scatter properties by flow cytometry. Representative histogram of αM staining (a) and quantification of expression levels as the geometric mean of fluorescence (GMF) from 4 (Cd44−/−) to 8 mice (wild type and Selplg−/−) per group. *, p<0.01; **, p<0.001, one-way ANOVA with Tukey's multi-comparison test (right panel).

FIG. 8a-d shows reduced nRBC capture in mice deficient in C3. (a) Interactions of RBC with adherent leukocytes were analyzed in wild-type and C3−/− mice, and mice were subsequently injected with 109 albumin-coated fluospheres to determine activation of the integrin αMβ2 on adherent leukocytes (d), as indicated in the Methods section and shown in FIG. 4. Panels (b) and (c), respectively, are representative micrographs of fluospheres bound to leukocytes in venules from wild-type and C3−/− mice. These experiments indicate that the reduction in RBC-leukocyte interactions in C3−/− mice is not due to reduced integrin activity; scale bar=10 μm; arrows indicate the direction of flow. n=27-35 venules from 4 mice per group; ***, p=0.0005, Mann-Whitney test.

FIG. 9a-c shows specificity of albumin-coated fluosphere capture in vivo. To rule out the possibility that fluospheres might bind to endogenous albumin and be uptaken non-specifically by phagocytes, we coated fluospheres with polyvinyl alcohol (PVA) which prevents binding of plasma albumin. Mice prepared for intravital microscopy were injected with (a) 109 albumin-coated fluospheres (short arrows) and APC-labeled anti-Gr-1 antibody (arrowhead) or (b) 109 PVA-coated red fluospheres (arrowheads) 180 min after TNF-α administration. After 10 min, images were acquired in the brightfield, FITC (short arrows) and Cy5 (panel (a)) or Cy3 (panel (b)) channels. Specific binding of fluospheres was restricted to Gr-1+ leukocytes. (c) The number of fluospheres bound to adherent leukocytes (95 beads scored from 2 mice) indicates that the presence of albumin is required for fluosphere capture. PVA-coated fluospheres present in the middle panel were free-flowing in the circulation and did not bind to leukocytes or the endothelium. Scale bars=10 μm. Large arrows indicate the direction of flow.

FIG. 10a-b shows that fluosphere capture by adherent leukocytes correlates with the level of αMβ2 expression. Mice prepared for intravital microscopy were injected i.v. with 1 μg APC-conjugated anti-αM, followed by 109 albumin-coated green fluospheres (short arrow) 180 min after TNF-α treatment. (a) Images were acquired in the brightfield, FITC (short arrow) and Cy5 (arrowhead) channels. (b) Analysis of relative αMβ2 (Mac-1) expression was performed using the SlideBook software by measuring the mean intensity of Cy5 fluorescence in adherent leukocytes and subtraction of background fluorescence present in an equivalent area in the plasma. The relative intensity associated to each cell was normalized to that of the leukocyte with the highest intensity of fluorescence for each mouse (100% expression). The number of fluospheres bound to the same cells was also counted. Data in the right plot is presented as mean±SEM, and was derived from the analysis of 93 leukocytes from 18 venules in 2 mice. Large arrow indicates the direction of flow. Scale bar=10 μm.

FIG. 11a-b shows in vivo detection of reactive oxygen species (ROS) by adherent leukocytes following anti-H2d infusion. Balb/c mice prepared for intravital microscopy were injected i.v. with 1 μg APC-conjugated anti-L-selectin and 1 μg PE-conjugated anti-CD41 180 min after TNF-α treatment. Mice were also injected with 29 nmoles of dihydroxyrhodamine-123, a probe uptaken by cells that is oxidized to fluorescent rhodamine-123 in the presence of intracellular ROS. (a) Images were acquired in the brightfield (BF), FITC (Rhodamine-123; arrowhead), Cy5 (L-selectin; small arrow with diamond arrowhead) and Cy3 (CD41; short arrow) channels, before and after injection of 3.5 mg/kg anti-H2d antibody. The frequency of ROS-producing leukocytes was calculated by determining the number of rhodamine-123-positive cells (asterisks in the merge+BF micrograph) out of all adherent leukocytes (2 of 11 in this particular image). Scale bar=10 μm. The arrow indicates the direction of the blood flow. (b) The frequency of ROS-producing leukocytes at different time periods after anti-H2d injection. A rapid activation of neutrophils is induced upon treatment and is sustained over time. Data obtained from 5 different experiments.

FIG. 12a-c shows N-acetyl-cysteine prevents ROS generation by adherent leukocytes and vascular permeability after anti-H2d infusion. (a) TNF-α-treated mice prepared for intravital microscopy were injected i.v. with 150 mg/kg n-acetyl-cysteine (N-AcC) together with the probe dihydroxyrhodamine-123 to determine ROS production by adherent leukocytes before and after injection of anti-H2d as indicated in FIG. 11. ***, p<0.0001, Mann-Whitney test; data obtained from the analysis of 20-33 venules from 3-4 mice. (b) FITC-dextran was injected at the end of the experiments to determine vascular permeability. Images were acquired in the FITC channel (FITC-Dextran; green with blue intensity masking) with a 10× objective. (c) Vascular permeability was determined by quantifying the amount of intravascular and total FITC-dextran signal in the micrographs (right panel). n=20-28 venular fields; **, p<0.01 compared to the other groups, Kruskal-Wallis test with Dunn's multigroup comparison. Vertical scale bar=50 μm.

FIG. 13a-b shows kinetics of leukocyte recruitment and RBC-leukocyte interactions in SCD mice. Analyses were performed before (surgical trauma only) and after TNF-α administration. Left panel shows the number of adherent leukocytes (WBC) in 100 μm-long venular segments. Right panel represents the number of sRBC interactions per adherent leukocyte per minute. Each dot represents one venule recorded at the indicated times.

FIG. 14 shows frequency of adherent leukocyte subsets in venules of sickle transgenic mice. Analyses were performed before (0-90 min) or at different times (91-180 and 181-270 min) after administration of TNF-α. Subsets were identified by high-speed MFIM using fluorescently conjugated CD45, Gr-1 and F4/80 antibodies. No statistical difference was found in the frequencies for the same leukocyte subsets at the different time points. PMN, neutrophils; Mono, monocytes; lymph, lymphocytes.

FIG. 15 shows leukocyte recruitment in venules of wild-type, Selp−/− and Sele−/− mice reconstituted with hematopoietic stem and progenitor cells from SCD mice. In some experiments, SCD mice were treated with 1 mg/kg of control or blocking antibodies to P- or E-selectin. Cremasteric venules of fully reconstituted mice (100% donor SCD) were analyzed by intravital microscopy at the times indicated. Bars represent mean values±SEM. n=7-17 mice per group. *, p<0.05; **, p<0.01; ***, p<0.001; §, p<0.01 compared to values at the same time period in the other groups; one-way ANOVA test with Bonferroni correction. “EC” denotes the endothelial cell phenotype or antibody treatment to inhibit endothelial selectins.

FIG. 16a-c shows effect of adhesion receptor deficiency and SCD mutation on αMβ2 activity on adherent leukocytes. (a) Wild-type and gene-targeted mice, or mice transplanted with (b& c) BM cells from SCD mice prepared for intravital microscopy were injected with albumin-conjugated fluospheres as indicated in Materials and Methods. The number of fluospheres associated to each adherent intravascular leukocyte was scored and the fraction of cells displaying very high αMβ2 activity (i.e., binding more than 8 beads) averaged and represented as bar graphs. (c) The frequency of leukocytes binding different fluosphere numbers is represented in the histogram . Data obtained from the analysis of n=227-507 cells from 4-6 mice per group. The fraction of leukocytes with elevated αMβ2 activity correlates with the frequency of RBC-leukocyte interactions. Bars represent mean±SEM.

FIG. 17 shows hemodynamic parameters of venules analyzed for the results presented in FIGS. 2 and 6, and FIG. 7. Values are mean±SEM. Shear rates in the sickle transgenic groups (lower table) correspond to the 181-270 min time periods. “Deaths” refers to the number of animals that have died in the course of the experimental period. Note the protection when E-selectin is absent or blocked. *, p<0.05 compared to the WT (top table), DMSO (middle table) or SS-WT group (bottom table) in each table; one-way ANOVA with Tukey's multigroup comparison test.

FIG. 18 shows hemodynamic parameters were analyzed from intravital microscopy recordings of venules used for the results shown in FIGS. 4 and 6. Presented are means±SEM of values per venule. *, p<0.05 compared to wild type or SA-WT one-way ANOVA with Tukey's multicomparison test.

### 5. DETAILED DESCRIPTION OF THE INVENTION

The present invention provides for methods for inhibiting or reducing inflammation-associated tissue damage, and particularly tissue and organ damage in the context of a transfusion reaction (especially Transfusion-Related Acute Lung Injury or “TRALI”) comprising inhibiting the activation of an E-selectin-mediated pathway, for example, but not limited to, E-selectin-mediated activation of Mac-1 (via ESL-1 and Src family members such as Src, Yes, Fyn, Fgr, Lck, Hck, Blk, Lyn and Frk) and subsequent generation of reactive oxygen species (“ROS”).

Without being bound to any particular theory, inflammation-associated tissue damage results, for example, when neutrophils located adjacent to blood vessel endotheliem undergo E-selectin-mediated activation of Mac-1 (the β2 integrin αMβ2), wherein Mac-1 expression at the leading edge of the neutrophils increases, allowing for the capture of circulating platelets by Mac-1 mediated interactions, resulting in the generation of oxidative species in the neutrophils that subsequently results in tissue and vascular damage, for example lung injury. Inactivation of E-selectin or αMβ2 can prevented generation of oxidative species and tissue injury associated with the inflammation.

For clarity and not by way of limitation, this detailed description is divided into the following sub-portions:
(i) definitions;
(ii) inhibitors of E-selectin-mediated activation of Mac-1;
(iii) methods of treatment; and
(iv) pharmaceutical compositions.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “inflammation” encompasses both acute responses (i.e., responses in which the inflammatory processes are active) as well as chronic responses (i.e., responses marked by slow progression and formation of new connective tissue).

The terms “vaso-occlusion” or “VOC” refer to the occlusion or restriction in lumen diameter of a blood vessel. In some embodiments, vaso-occlusion is associated with or caused by an inflammatory response. In other embodiments, vaso-occlusion is associated with or caused by traumatic injury to a blood vessel. In other embodiments, vaso-occlusion is associated with sickle cell disease.

The term “E-selectin” refers to a cell adhesion molecule expressed on endothelial cells. In one non-limiting embodiment, E-selection is activated by cytokines, for example, TNF-α. In other non-limiting embodiments, the E-selectin is specific for binding to glycoproteins, for example, E-selectin ligand-1, P-selectin glycoprotein ligand-1 and CD44. In certain embodiments, E-selectin is encoded by the Homo sapiens selectin E (SELF) gene (GenBank Acc. No. NM 000450). Alternatively, E-selectin can be encoded by any nucleic acid molecule exhibiting at least 50%, at least 60%, at least 70%, at least 80%, at least 90%, at least 95%, or up to 100% homology to the SELE gene (as determined by standard software, e.g. BLAST or FASTA), and any sequences which hybridize under standard conditions to these sequences.

The term “P-selectin” refers to a cell adhesion molecule expressed on endothelial cells. In one non-limiting embodiment, P-selectin is activated by cytokines, for example, TNF-α. In other non-limiting embodiments, P-selectin is specific for binding to glycoproteins, for example, E-selectin ligand-1, P-selectin glycoprotein ligand-1 and CD44. In other embodiments, P-selectin is encoded by the Homo sapiens selectin P (granule membrane protein 140 kDa, antigen CD62) (SELP) gene (GenBank Acc. No. NM_{—}003005). Alternatively, P-selectin can be encoded by any nucleic acid molecule exhibiting at least 50%, at least 60%, at least 70%, at least 80%, at least 90%, at least 95%, or up to 100% homology to the SELP gene (as determined by standard software, e.g. BLAST or FASTA), and any sequences which hybridize under standard conditions to these sequences.

The terms “E-selectin ligand-1” or “ESL-1” refer to a glycoprotein that can bind to E-selectin and P-selectin. In one embodiment, the ESL-1 is expressed by a leukocyte, for example, a neutrophil. In other embodiments, ESL-1 is encoded by the human GLG1 gene (GenBank Acc. Nos. NM_{—}001145667, NM_{—}001145666, and NM_{—}012201). Alternatively, ESL-1 can be encoded by any nucleic acid molecule exhibiting at least 50%, at least 60%, at least 70%, at least 80%, at least 90%, at least 95%, or up to 100% homology to the GLG1 gene (as determined by standard software, e.g. BLAST or FASTA), and any sequences which hybridize under standard conditions to these sequences.

The terms “Mac-1,” “Macrophage-1 antigen,” or “αMβ2” refer to a complement receptor, for example, a CR3 complement receptor, that can bind to C3b and C4b complement elements. In one embodiment, Mac-1 is a heterodimer integrin comprising CD11b (encoded by the human Integrin alpha M (ITGAM) gene, GenBank Acc. No. NM_{—}000632) and CD 18 (encoded by the human Integrin beta-2 (ITGB2) gene, GenBank Acc. Nos. NM_{—}000211 and NM_{—}001127491). Alternatively, CD11b and CD18 can be encoded by any nucleic acid molecule exhibiting at least 50%, at least 60%, at least 70%, at least 80%, at least 90%, at least 95%, or up to 100% homology to the ITGAM or ITGB2 genes, respectively, (as determined by standard software, e.g. BLAST or FASTA), and any sequences which hybridize under standard conditions to these sequences.

In one non-limiting embodiment, the binding of E-selectin to ESL-1 activates an Src kinase signal transduction pathway (e.g., via activation of the Src family members Src, Yes, Fyn, Fgr, Lck, Hck, Blk, Lyn and Frk), that activates an αMβ2 integrin in the neutrophil. According to one embodiment of the invention, the binding of ESL-1 to E-selectin induces polarized, activated αMβ2 integrin clusters at the leading edge of a neutrophil crawling, rolling or fixed along a blood vessel endothelium, wherein the αMβ2 integrin clusters facilitate the capture of erythrocytes and/or platelets (e.g., by binding to the erythrocytes and/or platelets) circulating in the blood. In one embodiment, capture of erythrocytes and/or platelets by the neutrophil results in the generation of reactive oxygen species (“ROS”) in the neutrophil.

In another non-limiting embodiment, the capture of sickle cell erythrocytes by αMβ2 microdomains leads to acute lethal vascular occlusions.

In other non-limiting embodiments, following transfusion, for example, a blood transfusion, polarized neutrophils undergo E-selectin mediated activation of αMβ2, and capture circulating platelets, resulting in the generation of reactive oxidative species that produces vascular damage and lung injury, such as, but not limited to, transfusion-related acute lung injury (TRALI).

The terms “P-selectin glycoprotein ligand-1” or “PSGL-1” refer to a glycoprotein that can bind to E-selectin and P-selectin. In one embodiment, the PSGL-1 is expressed by a leukocyte, for example, a neutrophil. In other embodiments, PSGL-1 is encoded by the human Selplg gene (GenBank Acc. No. NM_{—}003006). Alternatively, PSGL-1 can be encoded by any nucleic acid molecule exhibiting at least 50%, at least 60%, at least 70%, at least 80%, at least 90%, at least 95%, or up to 100% homology to the Selplg gene (as determined by standard software, e.g. BLAST or FASTA), and any sequences which hybridize under standard conditions to these sequences.

The term “CD44” refers to a glycoprotein that can bind to E-selectin and P-selectin. In one embodiment, the CD44 is expressed by a leukocyte, for example, a neutrophil. In other embodiments, CD44 is encoded by the human Cd44 gene (GenBank Acc. Nos. NM_{—}000610, NM_{—}001001389, NM_{—}001001390, NM_{—}001001391, and NM_{—}001001392). Alternatively, CD44 can be encoded by any nucleic acid molecule exhibiting at least 50%, at least 60%, at least 70%, at least 80%, at least 90%, at least 95%, or up to 100% homology to the Cd44 gene (as determined by standard software, e.g. BLAST or FASTA), and any sequences which hybridize under standard conditions to these sequences.

In other non-limiting embodiments, ESL-1, PSGL-1 and CD44 are glycoproteins that mediate recruitment of leukocytes to inflamed areas by a cascade of adhesive and signaling events that choreograph leukocyte migration. The glycoproteins mediate the physiological binding of leukocytes to P- and E-selectins, wherein leukocyte rolling along a blood vessel endothelium is followed by a first wave of activating signals from the endothelium that upregulates the function of integrin receptors, allowing firm arrest of neutrophil rolling.

According to the invention, a “subject” or “patient” is a human or non-human animal. Although the animal subject is preferably a human, the compounds and compositions of the invention have application in veterinary medicine as well, e.g., for the treatment of domesticated species such as canine, feline, and various other pets; farm animal species such as bovine, equine, ovine, caprine, porcine, etc.; wild animals, e.g., in the wild or in a zoological garden; and avian species, such as chickens, turkeys, quail, songbirds, etc.

In one embodiment, the subject or patient has been diagnosed with, or has been identified as having an increased risk of developing, tissue inflammation, for example, inflammation of blood vessel endothelium.

In other embodiments, the subject or patient has received, or has need diagnosed as needing, a transfusion, for example, a blood transfusion.

In other embodiments, the subject or patient has been diagnosed with, or has been identified as having, sickle cell disease.

### 5.2 Inhibitors of E-Selectin-Mediated Pathways

The present invention provides for methods for inhibiting inflammation-associated tissue damage, for example, but not limited to, tissue and organ damage in the context of a transfusion reaction (e.g., Transfusion-Related Acute Lung Injury or TRALI). According to one embodiment of the present invention, inhibiting the tissue damage comprises administering a compound to a subject or patient that inhibits activation of an E-selectin-mediated pathway. For Example, and not by way of limitation, the compound may inhibit E-selectin-mediated activation of Mac-1 (for example, by inhibiting E-selectin, ESL-1, Mac-1 or Src family members such as, for example, Src, Yes, Fyn, Fgr, Lck, Hck, Blk, Lyn and Frk), and subsequent generation of reactive oxygen species. In other embodiments of the invention, the generation of reactive oxygen species is inhibited directly.

In one non-limiting embodiment of the invention, the inhibitor is an antibody that specifically binds to and inhibits, blocks or reduces the activity of an element of an E-selectin-mediated pathway, for example, an E-selectin-mediated activation pathway of Mac-1. For example, the antibody may be specific for one or more of E-selectin, ESL-1, Mac-1 or an Src family member (for example, but not limited to, Src, Yes, Fyn, Fgr, Lck, Hck, Blk, Lyn and Frk). The antibody may be a monoclonal or a polyclonal antibody. In one non-limiting embodiment, the antibody is a human or humanized antibody. The antibody may be produced by any means known in the art.

In one non-limiting embodiment, the antibody is a humanized antibody or fragments thereof (for example, but not by way of limitation, Murine IgG2a produced by hybridoma Cat. #BMS110 of Bender MedSystems, murine IgG Cat. # HM4002 by Hycult Biotechnology by, a humanized antibody as described in WO1996/040942, or HuEP5C7).

In other non-limiting embodiments, the inhibitor is a molecule, compound or drug that inhibits, blocks or reduces expression of an element of an E-selectin-mediated pathway, for example, E-selectin-mediated activation of Mac-1. For example, the inhibitor may be an antisense or RNAi molecule that inhibits the expression of a gene, such as, but not limited to, one or more of the genes encoding E-selectin, ESL-1, Mac-1 or an Src family member (for example, but not limited to, Src, Yes, Fyn, Fgr, Lck, Hck, Blk, Lyn and Frk).

In other non-limiting embodiments, the inhibitor is a molecule, compound or drug that inhibits, blocks or reduces the functionality of an element of an E-selectin-mediated pathway, such as E-selectin-mediated activation of Mac-1. For example, the molecule, compound or drug may inhibit, block or reduce the functionality of E-selectin, ESL-1, Mac-1 or an Src family member (such as, but not limited to, Src, Yes, Fyn, Fgr, Lck, Hck, Blk, Lyn and Frk). For example, the inhibitor may be a small molecule inhibitor, including but not limited to A205804 (4-[(4-Methylphenyl)thio]thieno[2,3-c]pyridine-2-carboxa mide) and molecules as described in Ali et al., 2003, FASEB J express article 10.1096; Stewart et al., 2001, J. Medicinal Chem. 44:988-1002; and Zhu et al., 2001, J. Medicinal Chem. 44:3469-3487.

In other non-limiting embodiments, the inhibitor is a molecule, compound or drug that inhibits, blocks or reduces the activity of one or more member of the Src family, such as Src, Yes, Fyn, Fgr, Lck, Hck, Blk, Lyn and Frk. Non-limiting examples of such agents include CGP76030 (Novartis Pharmaceuticals, Basel, Switzerland), INNO-406 (CytRx, Los Angeles, Calif., USA), SU6656 (CalBiochem, La Jolla, Calif.), PP1 (4-amino-5-(4-methylphenyl)-7-(t-butyl)pyrazolo [3,4-d]pyrimidine), PP2 (4-amino-5-(4-chlorophenyl)-7-(t-butyl)pyrazolo[3,4-d]pyramidine), and Dasatinib (BMS 354825; Bristol-Myers Squibb, New York, N.Y.).

In other non-limiting embodiments, the inhibitor is a molecule, compound or drug that blocks, inhibits or reduces the actions of a reactive oxygen species. For example, the inhibitor may be an enzyme such as, for example, superoxide dismutases, catalases, glutathione peroxidases or peroxiredoxins; an antioxidant, such as ascorbic acid (vitamin C), tocopherol (vitamin E), uric acid, or glutathione; an ROS scavenger, such as n-acetyl-cysteine, DPI, diphenyleneiodonium chloride or polyphenol.

In other non-limiting embodiments, the inhibitor may be any molecule, compound or drug that inhibits, blocks or reduces the capture, binding or any other association, of a leukocyte, for example, a neutrophil, with an erythrocyte and/or a platelet.

In yet other non-limiting embodiments of the invention, the inhibitor is a molecule, compound or drug that reduces the amount or concentration of erythrocytes and/or platelets present in a patient's blood.

### 5.3 Methods of Treatment

In accordance with the invention, there are provided methods of using inhibitors of an E-selectin-mediated pathways, for example, E-selectin-mediated activation of Mac-1. The inhibitors used in the invention may be used to inhibit, block or reduce expression or activation of any element of an E-selectin-mediated pathway, for example, E-selectin-mediated activation of Mac-1, and thus reduce damage to a tissue or organ, for example, by blocking the generation of ROS. The methods of the invention may be used to inhibit and/or reduce inflammation and/or to treat (i.e., inhibit, block or reduce) tissue or organ damage related to inflammation, tissue or cellular trauma, transfusions or diseases of the blood, in a subject in need of such treatment. For example, the methods of the invention are useful for the treatment of conditions including, but not limited to, TRALI and sickle cell disease.

The present invention provides for methods of inhibiting and/or reducing inflammation and/or treating tissue or organ damage in a subject in need of such treatment by administration of a therapeutic formulation which comprises an inhibitor of an E-selectin-mediated pathway, for example, E-selectin-mediated activation of Mac-1. In particular embodiments, the formulation may be administered to a subject in need of such treatment in an amount effective to inhibit, block or reduce E-Selectin activation. In other non-limiting embodiments, the formulation may be administered to a subject in need of such treatment in an amount effective to inhibit, block or reduce Mac-1 activation, expression, or clustering at the leading edge of a neutrophil. In other embodiments, the the formulation may be administered to a subject in need of such treatment in an amount effective to inhibit, block or reduce the generation of ROS resulting from Mac-1 activation. Alternatively, the formulation may be administered to a subject in need of such treatment in an amount effective to inhibit, block or reduce the capture or association of a neutrophil with an erythrocyte and/or a platelet. Where the formulation is to be administered to a subject in vivo, the formulation may be administered systemically (e.g. by intravenous injection, oral administration, inhalation, etc.), or may be administered by any other means known in the art. The amount of the formulation to be administered may be determined using methods known in the art, for example, by performing dose response studies in one or more model system, followed by approved clinical testing in humans.

The present methods may be applied to a subject in need of such treatment. Suitable subjects include a subject who is suffering from an inflammatory condition, especially, but not limited to, a transfusion-associated inflammatory condition such as, but not limited to, TRALI. Other suitable subjects include those who, prior to transfusion, may be identified as being at particular risk of developing a transfusion-associated disorder, such as, but not limited to, subjects with a prior history of transfusion, subjects suffering from an inflammatory condition, subjects having sickle cell disease or trait, subjects in critical need of transfusion but where a suitably matched blood or blood product is not available, and other acutely ill subjects. Other inflammatory conditions that may be treated according to the invention include, but are not limited to, adult repiratory distress syndrome, arthritis, type I hypersensitivity, atopy, anaphylaxis, asthma, osteoarthritis, rheumatoid arthritis, septic arthritis, gout, juvenile idiopathic arthritis, still's disease, ankylosing spondylitis, inflammatory bowel disease, Crohn's disease or inflammation associated with vertebral disc herniation.

In certain non-limiting embodiments of the invention, an effective amount of an inhibitor of an E-selectin-mediated pathway, for example, E-selectin-mediated activation of Mac-1, is an amount which reduces the concentration or number of neutrophil cells located at a site of inflammation in a tissue or organ, for example, a blood vessel endothelium.

In other non-limiting embodiments of the invention, an effective amount of an inhibitor of an E-selectin-mediated pathway, for example, E-selectin-mediated activation of Mac-1, is an amount which reduces the concentration or level of Mac-1 expressed at the leading edge of a neutrophil.

In other non-limiting embodiments of the invention, an effective amount of an inhibitor of an E-selectin-mediated pathway, for example, E-selectin-mediated activation of Mac-1, is an amount which reduces the level or amount of plasma proteins present in a patient's lungs as assessed by broncho-alveolar lavage (BAL).

In other non-limiting embodiments of the invention, an effective amount of an inhibitor of an E-selectin-mediated pathway, for example, E-selectin-mediated activation of Mac-1, is an amount which reduces blood vessel occlusion in a patient, for example, a patient with sickle cell disease. In other non-limiting embodiments, an effective amount of an inhibitor of an E-selectin-mediated pathway, for example, E-selectin-mediated activation of Mac-1, is an amount which increases the diameter of a blood vessel lumen.

In other non-limiting embodiments of the invention, an effective amount of an inhibitor of an E-selectin-mediated pathway, for example, E-selectin-mediated activation of Mac-1, is an amount which reduces the generation of ROS by a neutrophil.

In other non-limiting embodiments of the invention, an effective amount of an inhibitor of an E-selectin-mediated, for example, E-selectin-mediated activation of Mac-1, is an amount which reduces inflammation in a blood vessel, tissue or organ.

In other non-limiting embodiments of the invention, an effective amount of an inhibitor of an E-selectin-mediated, for example, E-selectin-mediated activation of Mac-1, is an amount which reduces damage to lung tissue as measured by a test of pulmonary function, for example, but not limited to, tests of residual volume, gas diffusion tests, body plethysmography, inhalation challenge tests, exercise stress tests, spirometry, forced vital capacity (FVC), forced expiratory volume (FEV), forced expiratory flow 25% to 75%, peak expiratory flow (PEF), maximum voluntary ventilation (MVV), slow vital capacity (SVC), total lung capacity (TLC), functional residual capacity (FRC), expiratory reserve volume (ERV).

### 5.4 Pharmaceutical Compositions

The inhibitor compounds and compositions of the present invention may be formulated as pharmaceutical compositions by admixture with a pharmaceutically acceptable carrier or excipient.

In one non-limiting embodiment, the pharmaceutical composition may comprise an effective amount of an inhibitor of an E-selectin-mediated pathway, for example, E-selectin-mediated activation of Mac-1, and a physiologically acceptable diluent or carrier. The pharmaceutical composition may further comprise a second drug, for example, but not by way of limitation, an anti-inflammatory drug.

The phrase “pharmaceutically acceptable” refers to molecular entities and compositions that are physiologically tolerable when administered to a subject. Preferably, but not by way of limitation, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, or, for solid dosage forms, may be standard tabletting excipients. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

In a specific embodiment, the therapeutic compound can be delivered in a vesicle, in particular a liposome (see Langer, 1990, Science 249:1527-1533; Treat et al., 1989, in Liposomes in the Therapy of Infectious Disease and Cancer, Lopez-Berestein and Fidler eds., Liss: New York, pp. 353-365; Lopez-Berestein, ibid., pp. 317-327; see generally Lopez-Berestein, ibid.).

### 6. EXAMPLES

### Example 1

### Neutrophil Capture of Platelets Via E-Selectin-Induced Activation of the Integrin Mac-1

During high-speed multichannel fluorescence intravital microscopy (MFIM) experiments, neutrophils (identified by CD45+ Gr-1 +F4/80-expression) crawling on inflamed endothelium actively interact with circulating platelets (identified in vivo by CD41 expression). In conditions in which inflammation was induced by surgical trauma only, platelet interactions were relatively rare and mediated by both the TE (identified in vivo by L-selectin clustering) and LE (opposite to L-selectin clusters). By contrast, in venules inflamed by the administration of TNFα, platelet interactions with the LE of crawling neutrophils were markedly increased. Sickle erythrocyte interactions in inflamed venules occurred at the LE of neutrophils through E-selectin-induced activation of the integrin Mac-1 (Blood 110: 145; November 2007). The role of E-selectin and Mac-1 in platelet capture has been analyzed, and a marked reduction in captures at the LE in mice lacking E-selectin was observed, whereas these were virtually absent in mice deficient in Mac-1. These findings indicate that the increase in platelet capture during inflammation is the result of Mac-1 activation by endothelial E-selectin. Using a murine model of TRALI in which Balb/c mice are infused with high doses of anti-MHC-I antibodies (anti-H2d), the induction of lung injury (assessed by accumulation of plasma proteins in the broncho-alveolar lavage (BAL)) was found to correlate with a moderate thrombocytopenia and that depletion of platelets prior to anti-H2d infusion results in complete prevention of lung injury. Neutrophil-platelet interactions may therefore be required for TRALI. To assess this possibility, intravascular events was analyzed by MFIM of inflamed cremasteric venules before and after injection of anti-H2d. Platelet captures by crawling neutrophils were increased by 2-fold after anti-H2d antibody infusion, and this increase was prevented by blocking E-selectin. Infusion of anti-H2d markedly increased (9-fold) vascular permeability (measured by FITC-dextran leakage) that could be prevented by blocking E-selectin or Mac-1, or by depleting platelets. In vivo activation of crawling neutrophils after anti-H2d infusion was next assessed by analyzing the presence of reactive oxygen species (ROS) using the fluorescent probe dihydroxyrhodamine-123. It was found that ROS were rapidly induced in crawling leukocytes following anti-H2d infusion, and that this induction could be blunted by platelet depletion, indicating a requirement for heterotypic interactions during neutrophil activation. To determine whether observations made in the cremasteric circulation correlated with the pulmonary injury caused by anti-H2d infusion, protein accumulation in BAL fluids of mice pretreated with antibodies against E-selectin or Mac-1 was measured. Inhibition of either receptor strongly prevented the development of lung injury. In addition, n-acetyl-cysteine, a ROS scavenging molecule, completely prevented lung injury induced by anti-H2d. These results suggest that E-selectin-induced, Mac-1-mediated platelet capture by the LE of crawling neutrophils result in ROS production, which are responsible for the vascular and organ injuries observed in this model of TRALI. The heterotypic interactions described here share mechanisms with those triggering vaso-occlusive episodes in a humanized murine model of sickle cell disease, suggesting that they play important and previously unappreciated contributions to thrombo-inflammatory complications.

### Example 2

### Heterotypic Interactions Enabled by Polarized Neutrophil Microdomains Mediate Thrombo-Inflammatory Injury

Selectins and their ligands mediate leukocyte rolling allowing interactions with chemokines that lead to integrin activation and arrest. Here, it is demonstrated that E-selectin is critical to induce a secondary wave of activating signals transduced specifically by E-selectin ligand-1, that induces polarized, activated αMβ2 integrin clusters at the leading edge of crawling neutrophils, allowing the capture of circulating erythrocytes or platelets. In a humanized model of sickle cell disease (SCD), the capture of erythrocytes by αMβ2 microdomains leads to acute lethal vascular occlusions. In a model of transfusion-related acute lung injury, polarized neutrophils capture circulating platelets, resulting in the generation of oxidative species that produces vascular damage and lung injury. Inactivation of E-selectin or αMβ2 prevented tissue injury in both inflammatory models, suggesting broad implications of this paradigm in thrombo-inflammatory diseases. These results indicate that endothelial selectins can influence neutrophil behavior beyond its canonical rolling step through delayed, organ-damaging, polarized activation.

Leukocytes are recruited to inflamed areas by a tightly regulated cascade of adhesive and signaling events that choreograph their migration. The initial rolling step of neutrophils (PMNs) is mediated by endothelial P- and E-selectins¹⁻³. Three distinct glycoproteins, P-selectin glycoprotein ligand-1 (PSGL-1, encoded by the Selplg gene), E-selectin ligand-1 (ESL-1, encoded by Glg1), and CD44 (encoded by Cd44) mediate the physiological binding of murine PMNs to P- and E-selectins (encoded by Selp and Sele, respectively) through highly specialized contributions⁴. Leukocyte rolling is followed by a first wave of activating signals from the endothelium that upregulates the function of integrin receptors, allowing firm arrest. Although G-protein coupled receptor (GPCR)-induced signaling triggered by chemokines is the best characterized pathway, recent studies have uncovered additional signaling functions for E-selectin ligands (ESLs). For example, CD44-mediated signaling induces receptor redistribution to a major cluster on slowly rolling leukocytes⁴, whereas PSGL-1 induces the partial activation of αLβ2 integrin (LFA-1) on rolling leukocytes⁵. Although Glg1 sequence is homologous to the fibroblast growth factor receptor gene⁶, it remains unknown whether ESL-1 can transduce signals. In addition, E-selectin engagement can activate the other major β2 integrin αMβ2 (CD11b/CD18, Mac-1) on PMNs^{7,8}, but the actual ESL involved in this process has not been identified. αMβ2, a highly promiscuous receptor, can bind endothelial counter-receptors (ICAM-1 and -2), matrix and plasma proteins (fibrinogen and albumin)⁹, the complement fragment C3b¹⁰ and the glycoprotein GpIbα on platelets¹¹, thus contributing to leukocyte recruitment in inflamed vessels and platelet adhesion.

During their recruitment, leukocytes undergo rapid changes characterized by a marked molecular redistribution, a phenomenon referred to as polarization which has been mostly studied in lymphocytes¹². Polarized lymphocytes sort chemokine receptors¹³, activated β2 integrins¹⁴ and actin-remodeling GTPase clusters¹⁵ at the leading edge whereas the trailing edge (uropod) is enriched in heavily glycosylated proteins (e.g. PSGL-1, L-selectin, CD43 and CD44) and other components involved in membrane retraction^{12,15}. These specialized microdomains are thought to be important for the directional, chemokine-driven movement of leukocytes within blood vessels and across the endothelium.

Activated neutrophils (PMNs) recruited to inflamed areas mediate acute and chronic organ injury, as their infusion or increased number in circulation are sufficient to promote organ damage, while their depletion can curb it in multiple experimental or clinical settings¹⁶⁻¹⁸. In sickle cell disease (SCD), repeated cycles of polymerization and de-polymerization of the mutated β-globin alter the sickle erythrocyte (sRBC) membrane, causing a pro-inflammatory phenotype that promotes acute vaso-occlusive (VOC) episodes^{19,20}. Previous studies in a humanized murine model of SCD have shown that PMNs can capture circulating sRBC and that the rate of these interactions correlates with reductions in microcirculatory blood flow and death of the animals^{21,22}, but the molecular basis of these interactions has not yet been elucidated. SCD patients and other acutely ill individuals requiring blood transfusions are at risk of transfusion-related acute lung injury (TRALI), the most prevalent cause of transfusion-associated morbidity, where antibodies against the recipient's PMNs or other bioactive mediators from the transfused blood can elicit organ injury^{23,24}.

Here, it is demonstrated that the generation of activated integrin microdomains at the leading edge of PMNs drives vascular damage in TRALI and SCD through heterotypic interactions. We show using real time high-speed multichannel fluorescence intravital microscopy (MFIM) that firmly adherent PMNs undergo a delayed secondary wave of activating signals emerging from E-selectin expressed in inflamed endothelium, transduced into PMNs by ESL-1, and leading to the formation of active αMβ2 integrin microdomains at the leading edge. We show that this paradigm is central in the pathogenesis of thrombo-inflammatory diseases using SCD and TRALI mouse models.

### Results

### Heterotypic Interactions Mediated by Adherent Leukocytes are Induced During Inflammation

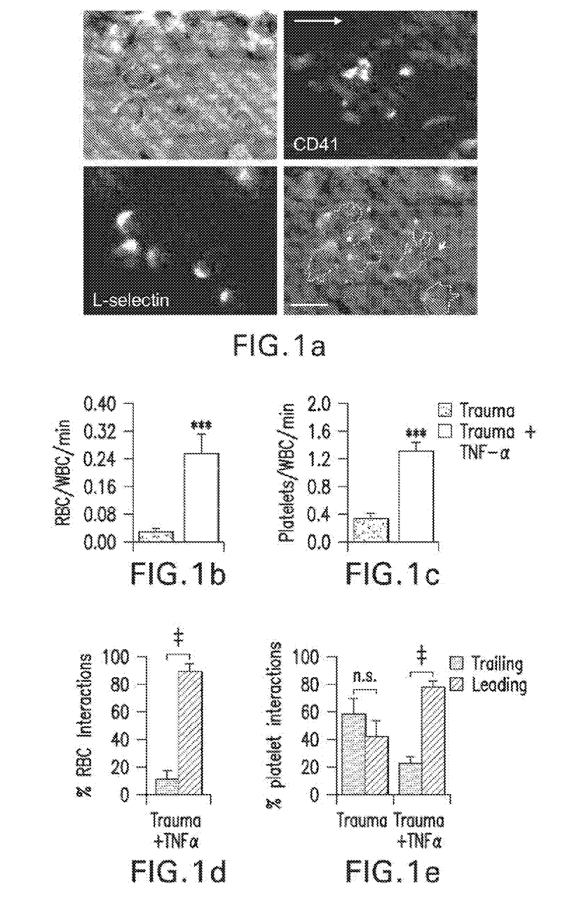

The cremasteric vasculature of wild-type mice after surgical trauma or following the administration of TNF-α was examined. Brightfield intravital microscopy revealed frequent interactions between adherent leukocytes and red blood cells carrying normal hemoglobin (nRBC) in cytokine-activated venules (FIG. 1a-b). These interactions, which tend to occur in venules with relatively low shear rates (<500 s⁻¹), can last up to several seconds. Endogenous platelets, identified by in vivo labeling with an anti-CD41 antibody²⁵ (FIG. 1a), were also found to interact with adherent leukocytes in venules after surgical manipulation, but these interactions were sharply enhanced after TNF-αtreatment (FIG. 1c). The leukocyte microdomains involved in these interactions were mapped by high-speed MFIM using clustered L-selectin expression as a marker of the trailing edge²⁶. nRBC interactions were mediated almost exclusively by the leading edge of adherent leukocytes, away from the L-selectin cluster (FIG. 1d). In contrast, platelet interactions with leukocytes adherent in non-inflamed venules (stimulated with surgical trauma alone) were mediated by both the trailing and leading edges (FIG. 1e). However, after cytokine stimulation, the marked increase in platelet interactions was mediated by the leading edge (FIG. 1e). These results indicate differential contributions of leukocyte microdomains in mediating interactions with blood components under inflammatory conditions.

Heterotypic Interactions at the Leading Edge are Regulated by E-Selectin/ESL-1 and Mediated by the Leukocyte Integrin Mac-1

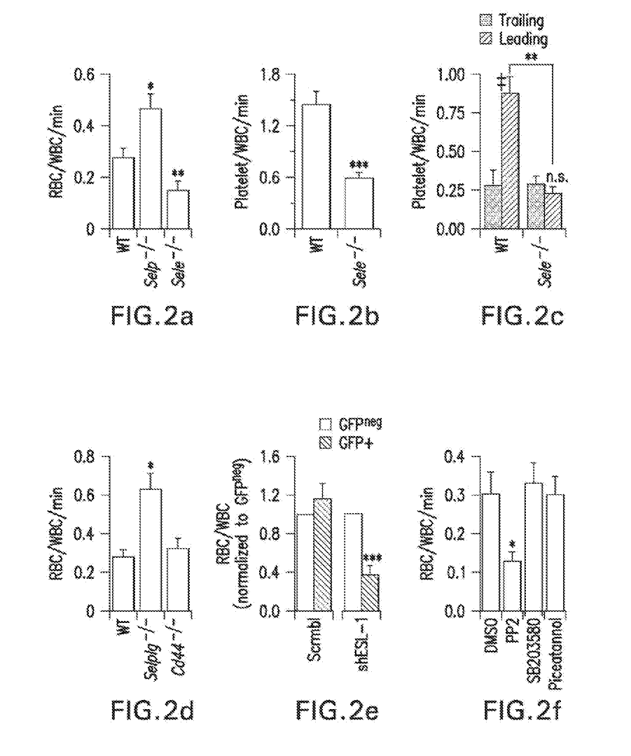

In SCD, leukocytes play a direct role in VOC since the absence of both endothelial selectins prevents leukocyte accumulation in inflamed venules, and protects SCD mice from VOC²². The individual roles of each endothelial selectin in the capture of nRBCs was investigated. As shown in FIG. 2a, the rate of nRBC interactions per adherent leukocyte were significantly reduced when E-selectin, but not P-selectin was absent. A similar reduction was found in Sele^{-/-} mice for platelet captures by adherent leukocytes (FIG. 2b), and mainly affected those mediated by the leading edge (FIG. 2c). Since E-selectin expression is restricted to the vascular endothelium, these results suggest that signals emanating from the endothelial cells, transmitted by ESLs into leukocytes, must regulate these heterotypic interactions.

Recent studies have revealed that PSGL-1, ESL-1, and CD44 comprise virtually all selectin binding activity on mouse PMNs⁴. Analyses of the behavior of circulating RBCs in Selplg^{-/-} or Cd44^{-/-} mice revealed no significant reduction in RBC captures compared to wild-type animals (FIG. 2d). Analyses of mice reconstituted with hematopoietic cells transduced with a lentiviral vector carrying a short hairpin RNA interference vector against Glg1, but not a scrambled control⁴, revealed a marked reduction in nRBC interactions with transduced leukocytes compared to those untransduced in the same venules (FIG. 2e). These results suggest that ESL-1 is the E-selectin ligand that mediates the activating signals allowing the heterotypic interactions during inflammation. Further, inhibition of Src kinases, but not p38 MAPK or the spleen tyrosine kinase (Syk) led to a reduction in nRBC-leukocyte interactions (FIG. 2f) comparable to that found in Sele^{-/-} mice or mice in which Glg1 is knocked-down, suggesting a role for Src kinases in transducing these activating signals.

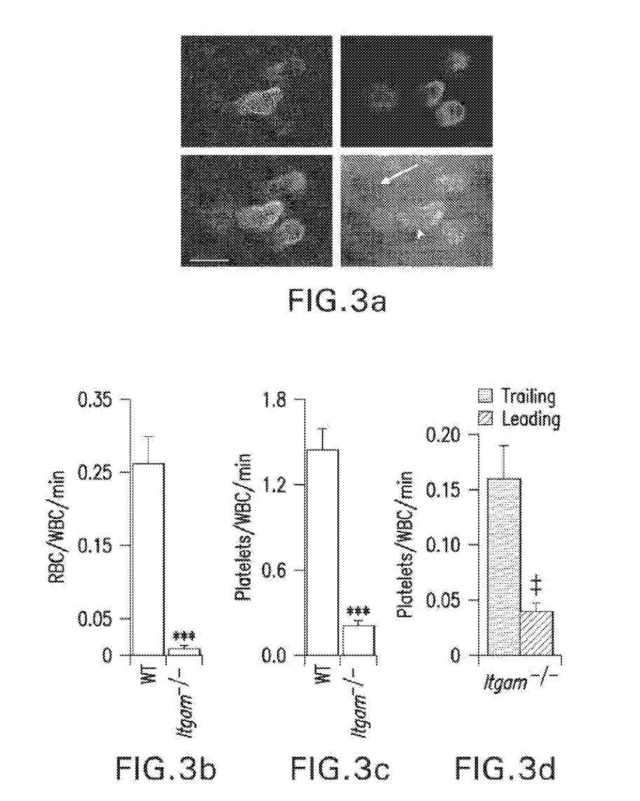

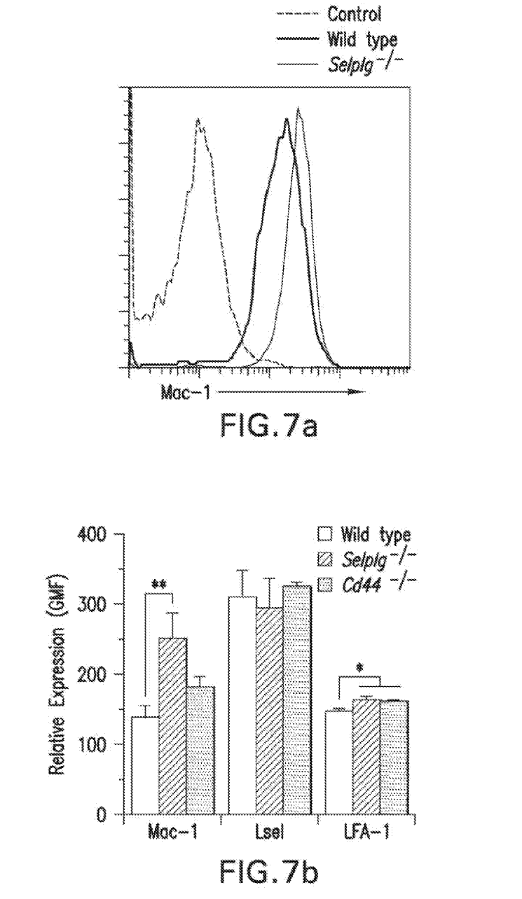

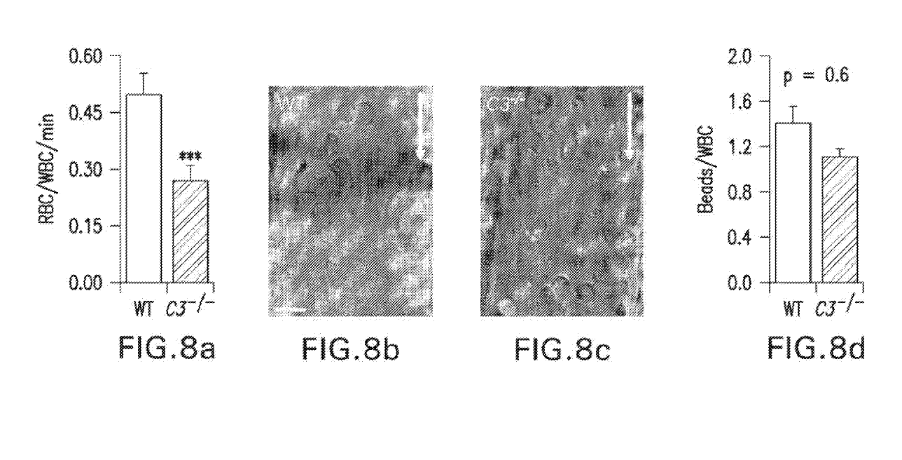

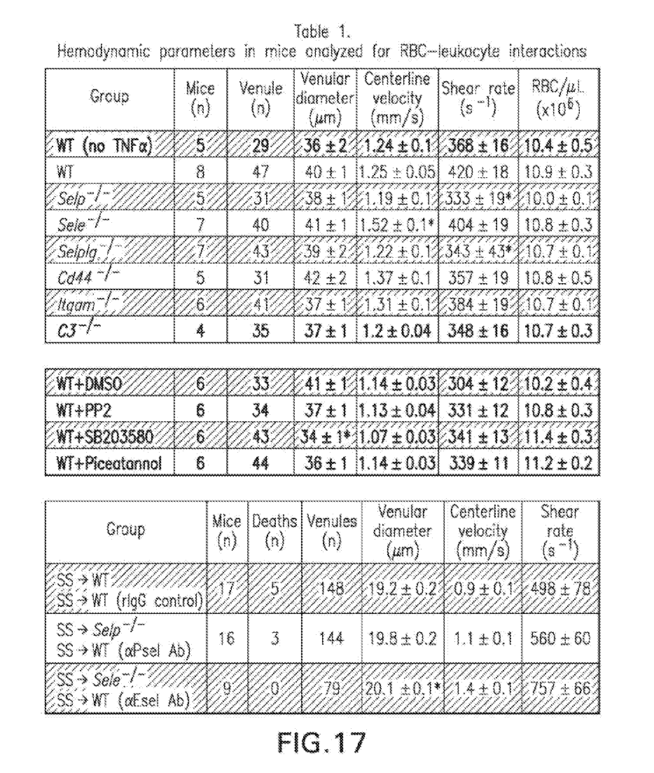

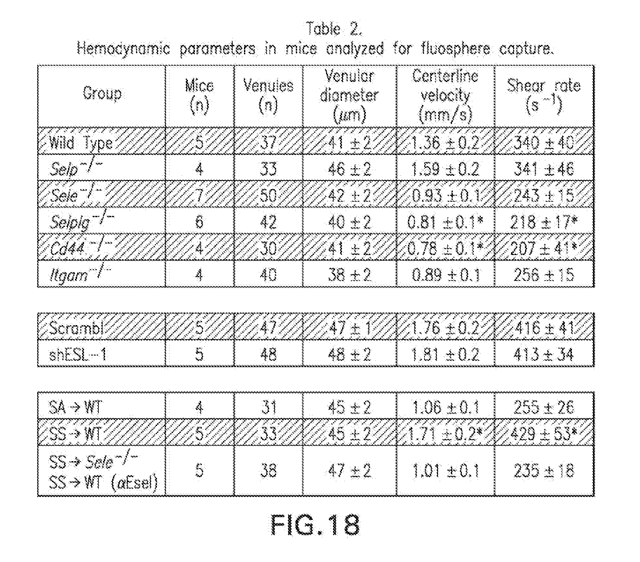

Activated β2 integrins have been reported to localize at the leading edge of adherent leukocytes¹⁴, and their activity can be modulated by E-selectin and Src kinases^{5,7,8,27,28}. We thus hypothesized that αMβ2 might be the receptor mediating these heterotypic interactions. High-speed MFIM analyses revealed that αMβ2 was homogeneously expressed on the surface of adherent leukocytes, including areas interacting with RBCs (FIG. 3a). We observed a dramatic reduction in the number of nRBC interacting with leukocytes deficient in αM (encoded by the Itgam gene; FIG. 3b) despite similar RBC counts among all groups (FIG. 17). In agreement with this finding, Selplg^{-/-} leukocytes, which exhibit increased nRBC interactions (FIG. 2d), expressed higher levels of αMβ2 (FIG. 7). Interestingly, mice deficient in the C3 complement protein, a ligand for αMβ2¹⁰, exhibited a significant, but partial, reduction in the frequency of nRBC interactions (FIG. 8), suggesting that complement opsonization of nRBCs is one mechanism by which RBC are captured. A dramatic reduction in platelet interactions was also observed with Itgam^{-/-} leukocytes (FIG. 3c). The reduction in platelet captures was not as marked as that of nRBCs, likely due to integrin-independent interactions at the trailing edge (FIG. 3d). These data indicate that the integrin αMβ2 is the receptor mediating heterotypic interactions at the leading edge of adherent leukocytes and that its activity is increased during inflammation.

### ESL-1 Regulates αMβ2 Activity on Neutrophil Microdomains

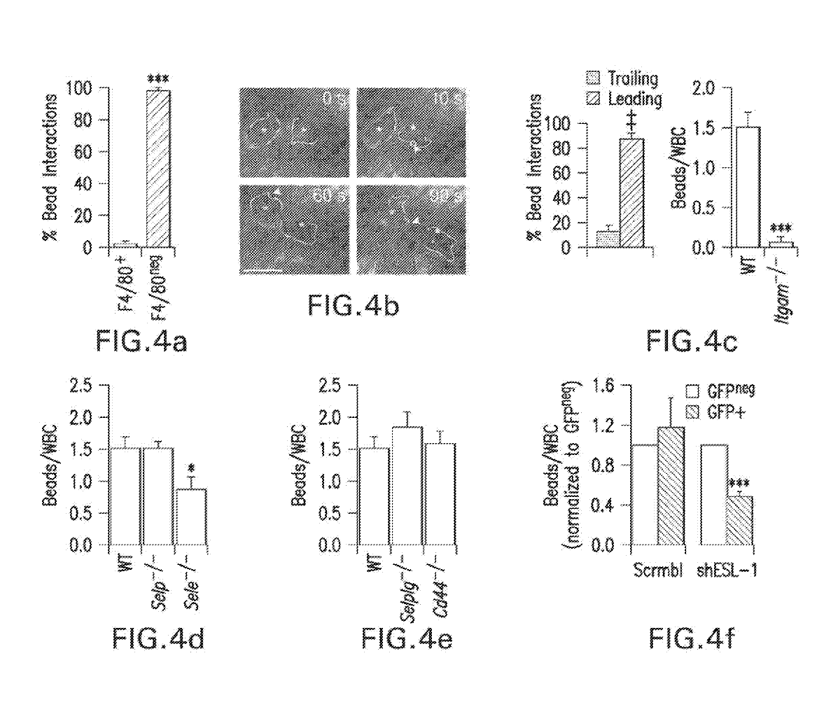

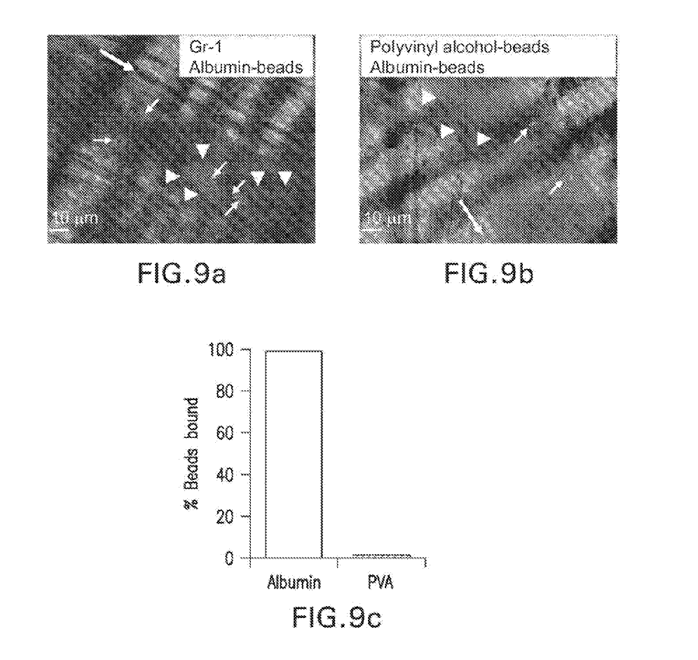

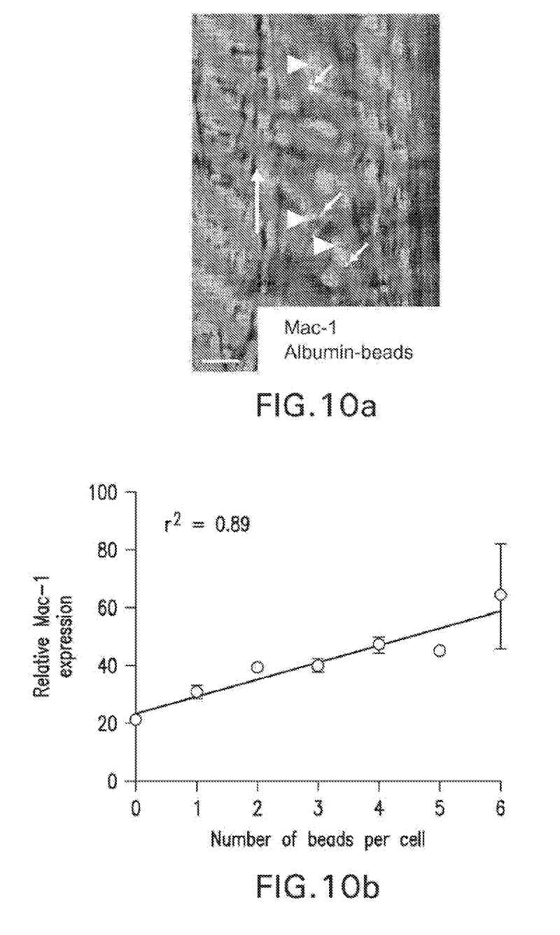

The aforementioned observations suggest that E-selectin/ESL-1 controls the activation of αMβ2 on adherent leukocytes. To assess this hypothesis, we have developed a novel in vivo assay to determine αMβ2 activity on adherent leukocytes in real time. We took advantage of a previously demonstrated property of denatured albumin to bind to leukocytes in a □2 integrin-dependent manner²⁹. We coated fluorescent beads (fluospheres; 1 μm in diameter) with albumin and tracked their behavior by high-speed MFIM after intravenous injection into mice treated with TNF-α. Albumin-coated fluospheres bound exclusively Gr-1^{pos} leukocytes, of which the vast majority were PMNs (Gr-1^{pos}F4/80^{neg}; FIG. 4a). Binding was specific since fluospheres coated with polyvinyl alcohol were not captured by leukocytes (FIG. 9). Fluosphere binding was mediated by the leading edge of adherent PMNs (FIG. 4b) via αMβ2; binding to Gr-1^{pos} leukocytes was virtually absent in Itgam^{-/-} mice (FIG. 4c). In addition, a strong correlation was found between αMβ2 protein expression in vivo and avidity (fluosphere capture) (FIG. 10).

We next used the fluosphere binding assay to determine whether E-selectin engagement could modulate αMβ2 activity in vivo. We found that the number of fluospheres captured by adherent leukocytes in Sele^{-/-} venules was significantly reduced compared to wild-type controls or Selp^{-/-} venules (FIG. 4d). Further, while fluosphere binding was not reduced in mice lacking PSGL-1 or CD44 (FIG. 4e) a marked reduction was found in leukocytes transduced with the shESL-1 vector (FIG. 4f). These results indicate that ESL-1 regulates regional αMβ2 avidity on the surface of adherent PMNs in vivo.

### E-Selectin-Induced αMβ2-Mediated Platelet-Neutrophil Interactions Generate Oxygen Radicals that Produce Organ Injury In TRALI

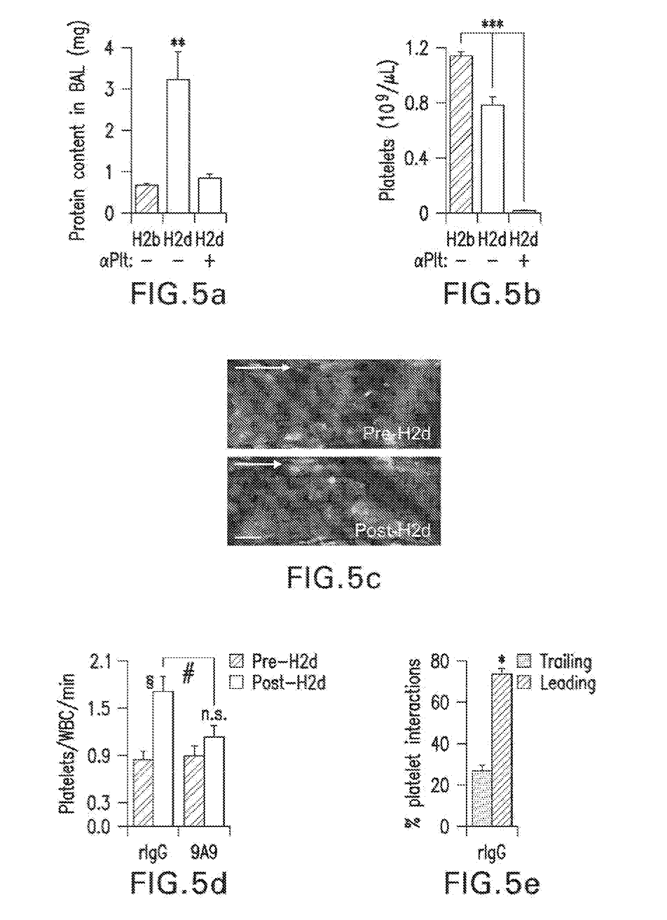

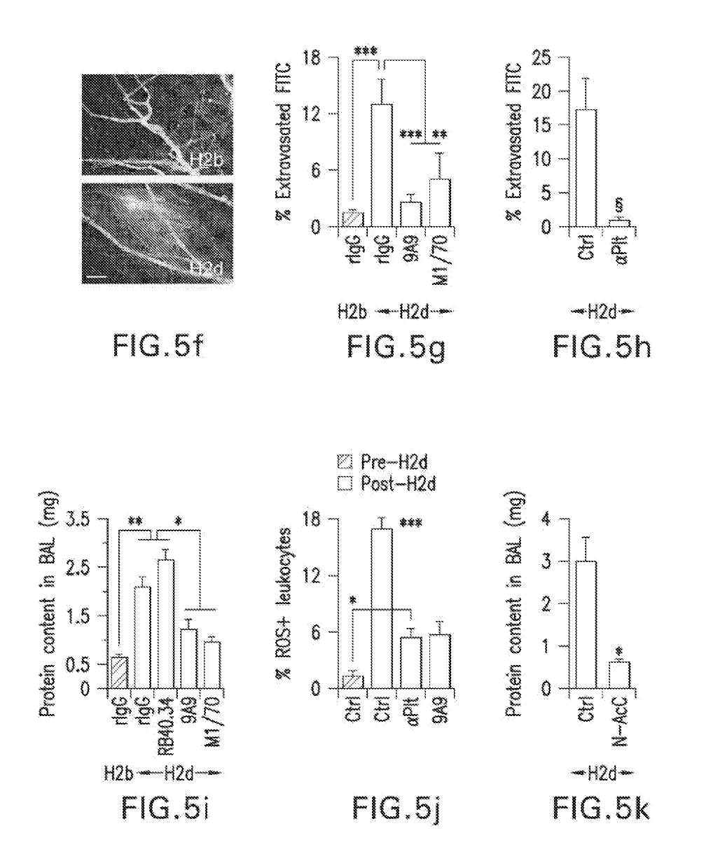

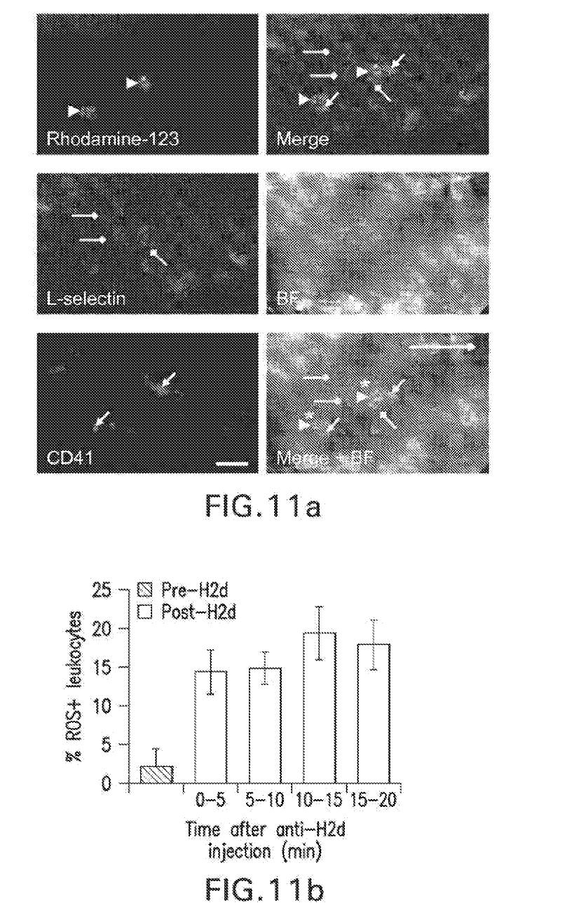

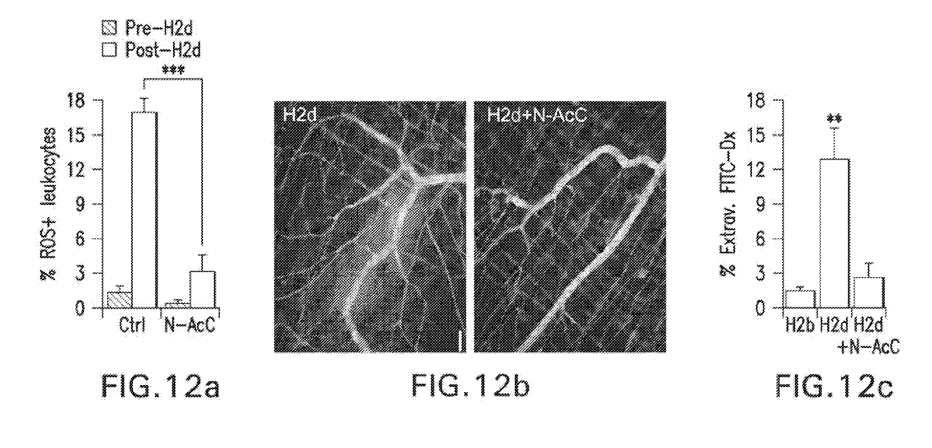

Whether the induction of heterotypic interactions through this mechanism might underlie the vascular injury that characterizes acute inflammatory processes was next inestigated. Intravenous injection of an anti-MHC-I antibody (haplotype H2d) into Balb/c mice has been used to model TRALI, a process that requires the presence of PMNs¹⁸. We induced lung injury in Balb/c mice by intravenous injection of an anti-H2d antibody as assessed by protein accumulation in the alveolar spaces (FIG. 5a). A moderate thrombocytopenia developed in these mice (FIG. 5b), suggesting that platelets might be involved in the pulmonary pathogenesis in this model. Platelet depletion prior to anti-H2d administration indeed resulted in complete abrogation of lung injury (FIG. 5a-b), without affecting PMN recruitment to the lungs (1.3±0.2×10⁵ vs. 1.1±0.1×10⁵ PMNs per mg lung tissue in control and platelet-depleted mice, respectively; n=4 mice; p=0.2). To gain further insight into the intravascular events involved in vascular injury, we used high-speed MFIM to observe heterotypic interactions in the cremasteric circulation in mice pre-treated with TNF-α before and after anti-H2d administration. Anti-H2d treatment significantly increased the number of platelet interactions with the leading edge of adherent leukocytes (FIG. 5c-e), which were inhibited by anti-E-selectin antibody treatment (FIG. 5d). Further, anti-H2d markedly increased the permeability of cremasteric venules, as determined by extravasated FITC-dextran (FIG. 5f-g), and this was prevented when platelets were depleted prior to antibody treatment (FIG. 5h). To determine the role of E-selectin-induced αMβ2 activation, other groups of mice were treated with antibodies against E-selectin (9A9) or αMβ2 (M1/70) prior to the anti-H2d challenge. It was found that vascular permeability was markedly reduced when either E-selectin or αMβ2 were inhibited (FIG. 5g). Furthermore, blocking E-selectin or αMβ2, but not P-selectin, strongly reduced lung injury (FIG. 5i). To investigate the potential role of tissue-damaging reactive oxygen species (ROS), a fluorescent probe was used to evaluate ROS formation by adherent neutrophils using MFIM. Minutes after anti-H2d infusion we found a dramatic increase in the frequency of ROS-producing adherent leukocytes (FIG. 5j and FIG. 11). Platelet depletion or E-selectin inhibition prior to anti-H2d treatment markedly reduced the number of ROS-producing leukocytes (FIG. 5j). In agreement with these observations, pretreatment with n-acetyl-cysteine, a ROS-scavenging molecule, completely prevented the vascular permeability (FIG. 12) and lung injury induced by anti-H2d (FIG. 5k). Taken together, the data suggest that E-selectin-induced αMβ2 activation mediates platelet capture at the leading edge of neutrophils, and that αMβ2 engagement, in turn, triggers the generation of ROS that produce vascular and organ injury.

### E-Selectin Induces Sickle RBC-Neutrophil Interactions and Vaso-Occlusion in a Humanized Mouse Model of SCD

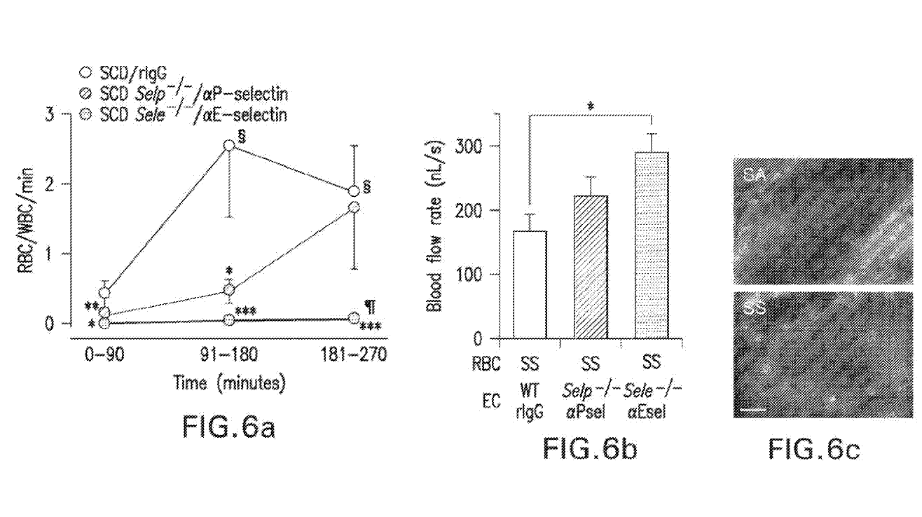

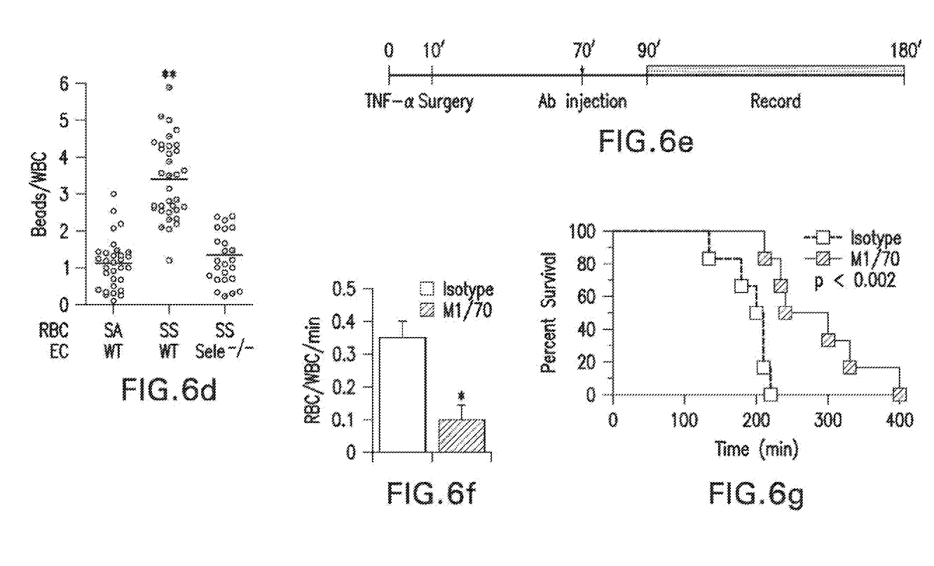

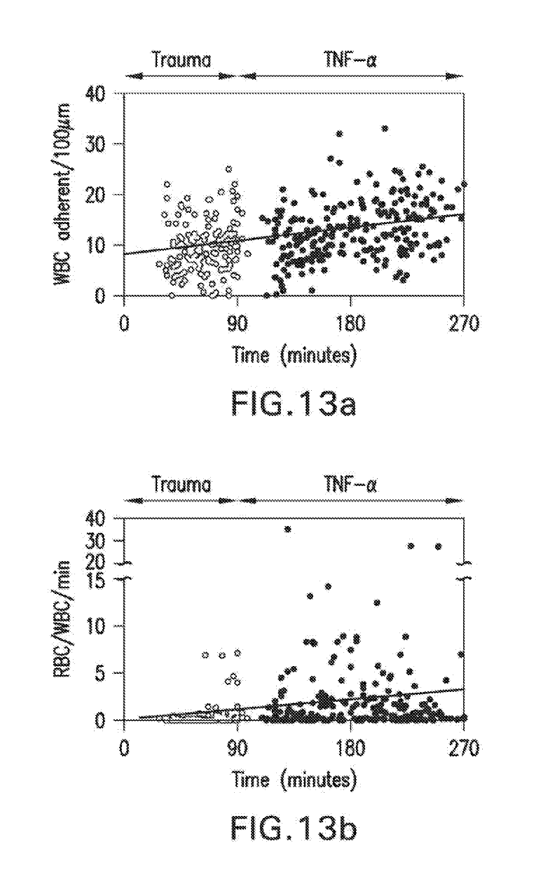

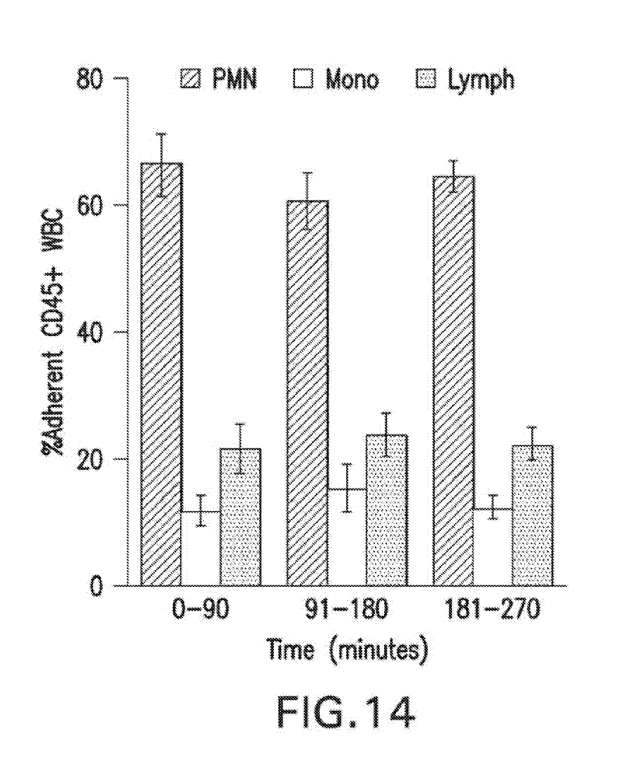

The interactions between sRBCs and adherent leukocytes can trigger VOC and death in a humanized model of SCD²². In SCD mice, the surgical trauma induced a robust recruitment of leukocytes in venules in the first 90 min after surgery (FIG. 13). Recordings of the same venules after systemic administration of TNF-α, 91-180 and 181-270 min after surgery, revealed further increase in leukocyte recruitment (FIG. 13). A fraction of circulating sRBCs were captured by adherent leukocytes and the rate of interactions per leukocyte increased by the administration of TNF-α (FIG. 6a and FIG. 13), eventually leading to reductions of blood flow in the cremasteric microcirculation and vital organs, often leading to the animal's death²². Notably, the rate of RBC-leukocyte interactions was significantly elevated in SCD mice compared to control animals (1.88±0.66 vs. 0.26±0.04 interactions per leukocyte per min at >90 min after TNF-α injection, p<0.001). Using high-speed MFIM, we previously identified PMNs as the leukocyte subset mediating the vast majority of sRBC captures in SCD²⁶. The induction of sRBC-leukocyte interactions after cytokine administration however was not due to the recruitment of more neutrophils at later times because the proportion of adherent CD45^{pos} Gr-1^{pos} F4/80^{neg} PMNs, relative to adherent lymphocytes and monocytes, did not significantly change during the three viewing periods (FIG. 14).

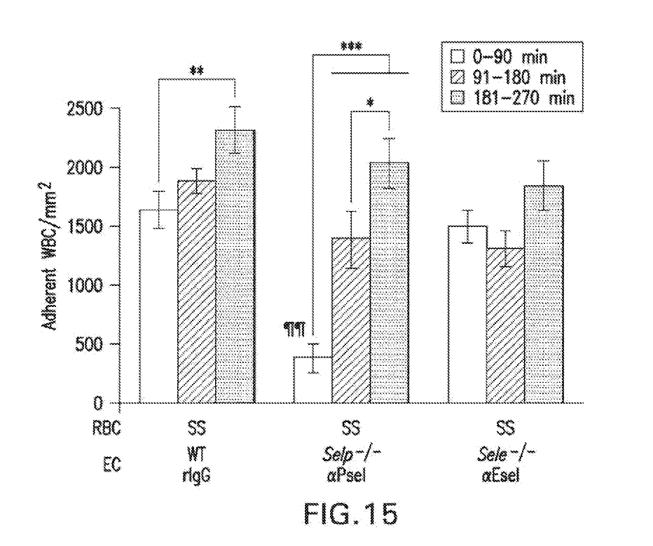

To determine whether E-selectin was involved in the increased frequency of heterotypic interactions observed in sickle mice after TNF-α administration, SCD mice that lacked endothelial E-selectin or P-selectin were generated by bone marrow transplantation, or these adhesion molecules were blocked using monoclonal antibodies. While the number of adherent leukocytes recruited in the three viewing periods were similar to control SCD mice (FIG. 15), the number of sRBC interactions per leukocyte was dramatically reduced in the absence of functional E-selectin (FIG. 6a), resulting in significant improvement in blood flow in the microcirculation (FIG. 6b). Moreover, the absence of functional E-selectin improved the survival of SCD mice during the experimental period [mortality in SCD mice: 31% (5/16) vs. SCD mice with Sele^{-/-}endothelium: 0% (0/9)]. In contrast, absence or inhibition of P-selectin had a partial effect, with the number of interactions, flow rates and survival similar to those of SCD control mice at the later time points (FIG. 6a-b; mortality: SCD mice with Selp^{-/-} endothelium: 18% (3/16)).

### Elevated αMβ2 Activity on SCD Adherent Leukocytes Mediates VOC

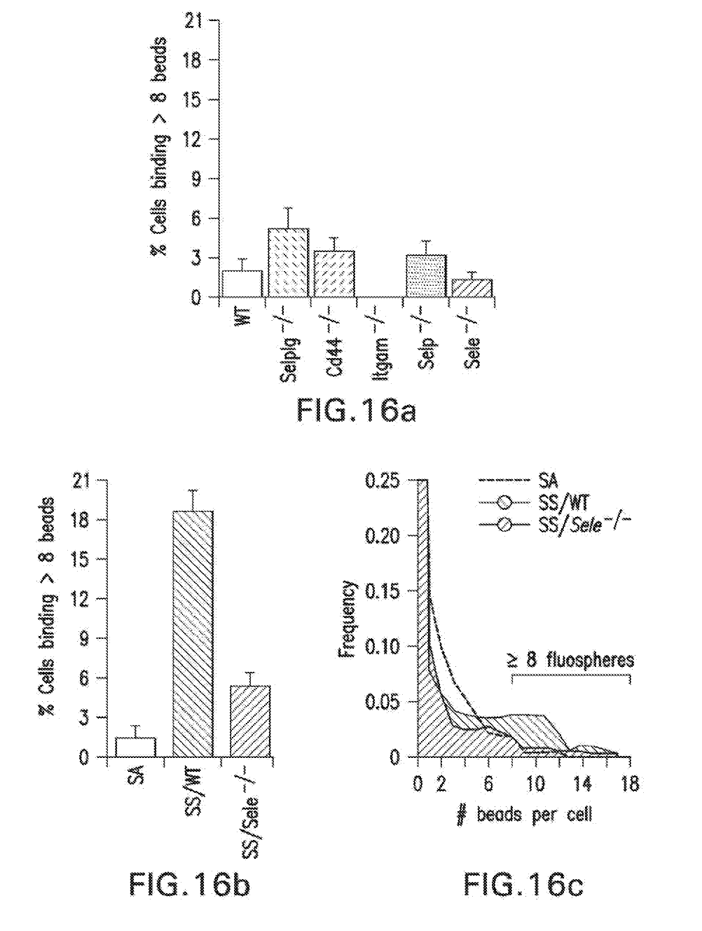

The increased rate of sRBC captures in SCD mice compared to nRBC interactions in wild-type mice suggests that αMβ2 activity is upregulated on adherent PMNs in SCD mice. To investigate this possibility, we analyzed αMβ2 activity in real time on SCD adherent leukocytes using the in vivo fluosphere binding assay (FIG. 6c). The number of captured beads was markedly elevated in SCD mice compared with control hemizygous animals (3-fold increase, p<0.0001; FIG. 6c-d). Further, frequency histograms revealed that this difference was largely due to the presence of a subset of leukocytes exhibiting marked integrin activation since 18% of adherent leukocytes in SCD mice captured >8 beads compared to 1.5% in hemizygous animals (FIG. 16). Moreover, in the absence of E-selectin in SCD mice, αMβ2 activity on adherent leukocytes was reduced to levels similar to those found in control hemizygous animals (FIG. 6d). These results further emphasize the critical role of E-selectin in regulating regional αMβ2 activity on adherent PMNs.

To test whether αMβ2 mediates sRBC-leukocyte interactions, SCD mice were injected i.v. with 1 mg/kg of anti αMβ2 or isotype control antibody 70 min after injection of TNF-α (FIG. 6e). Anti-αMβ2 administration reduced the interactions between sRBCs and leukocytes by 79%, compared with control antibody (FIG. 6f). Further, anti-αMβ2 treatment significantly increased the wall shear rates (740±62 s⁻¹ in isotype control vs 933±58 s⁻¹ in M1/70-treated mice; p<0.05) and significantly prolonged survival after TNF-α treatment (FIG. 6g). These data indicate that E-selectin expressed during the inflammatory response triggers regional activation of leukocyte αMβ2 that promotes sRBC interactions with intravascular leukocytes and VOC in SCD mice.

### Discussion

Intravascular accumulation and activation of PMNs to localized inflamed areas can result in vascular and organ damage. Beyond its role in promoting the recruitment of inflammatory leukocytes, here it is shown that the endothelium sends activating signals that are critical for vascular injury. We have found that these E-selectin-mediated signals are specifically transduced via ESL-1 and result in local activation of the integrin αMβ2 at the leading edge of crawling neutrophils. Luminal, activated αMβ2 clusters mediate heterotypic interactions with circulating RBCs and platelets, which can produce vascular occlusion or damage. The protection from organ injury or death by targeting this pathway in two distinct disease models suggests that this paradigm may have broad implications in other thrombo-inflammatory diseases.

That leukocytes require activating signals to arrest on the endothelium has been well established experimentally. The best characterized pathway is that mediated by GPCRs which upregulate integrin binding activity on rolling leukocytes^{30,31}. Much less is known however, about the events taking place after leukocytes have adhered. Recent improvements in imaging technologies have revealed a remarkable dynamism and communion between the leukocyte and the endothelium where raised endothelial membrane projections, enriched in integrin counterreceptors, embrace leukocytes as they crawl on the endothelial surface^{32,33}. High-speed MFIM has revealed that at least two-thirds of adherent myeloid leukocytes (Gr-1^{pos}) are actively migrating, presumably searching for pathogens or a site of extravasation²⁶. Further, virtually all adherent leukocytes exhibit a polarized appearance in vivo with clustered L-selectin at the uropod²⁶. This marker was used in the present study to map the heterotypic interactions at the leading edge of PMNs during inflammatory conditions, and show that although αMβ2 integrin was expressed homogenously on the cell surface, its activity was specifically enhanced at the leading edge of crawling leukocytes. Leukocyte polarity is critical for their migration as cells must reorganize chemokine receptors, integrins, and various signaling and cytoskeletal constituents for directional migration¹⁵. The results uncover a novel non-migratory function for leukocyte polarization in which clustered activation of an integrin is directly involved in disease pathogenesis through the generation of intravascular heterotypic interactions.

In vitro studies have previously shown that endothelial selectins can transduce signals into leukocytes^{7,29,34,35}. In vivo, PSGL-1 signaling through Syk can activate αLβ2 to an intermediary affinity confirmation that favors slow rolling on ICAM-1⁵, whereas CD44 can induce receptor clustering on slow rolling leukocytes through p38 signalilng⁴. Here, we show that a third physiological selectin ligand, ESL-1, is clearly a functional signaling receptor but that these signals are transmitted, unexpectedly, much later during the inflammatory cascade. Our studies suggest that these signals are transduced by Src kinases and affect the behavior of leukocytes that have adhered firmly, resulting in regional activation of αMβ2. These observations highlight the signaling diversity of ESLs that produces distinct activating phenomena in neutrophils. While P-selectin can also activate αMβ2 through Src kinases at early stages of leukocyte recruitment to favor adhesion³⁶, we show that this selectin is not required for the induction of heterotypic interactions in leukocytes that have already arrested on the endothelium. Thus, the timing of αMβ2 activation may dictate the neutrophil response in inflamed venules.

The present study also underscores the bidirectional signaling capacity of αMβ2 integrin. Our results indicate that ESL-1 can trigger “inside-out” αMβ2 activation at the leading edge of PMNs, while in turn the engagement of platelets by activated αMβ2 can trigger “outside-in” signals generating ROS^{37,38} that likely produce vascular damage in a model of acute lung injury triggered by anti-MHC-I antibodies. Engagement of αMβ2 has been shown to induce granular release and elicit vascular damage in a model of hemorrhagic vasculitis¹⁷. Interactions of platelets with neutrophils through P-selectin and PSGL-1 may also contribute to lung injury after acid challenge³⁹, raising the intriguing possibility that different stimuli may promote inflammation through distinct leukocyte microdomains. Since the infusion of the anti-H2d antibody causes vascular injury outside the lung¹⁸, we have used high-speed MFIM in the cremasteric microcirculation as a platform to obtain mechanistic insight. These analyses are consistent with a model whereby E-selectin is necessary to initiate a second wave of activation that upregulates αMβ2 locally, at the leading edge of neutrophils, allowing platelet capture. Platelets bound to αMβ2 then induce the formation of ROS by neutrophils which causes vascular damage. All key players in this model were verified in the lung injury model, suggesting that the MFIM observations in the cremasteric microcirculation likely reflect mechanisms at work in the lung.

Complement C3 was identified as one of the physiological αMβ2 ligands involved in RBC interactions, possibly through coating of aged or damaged RBCs⁴⁰. Elevated levels of C3-bound to deoxygenated sRBC or RBC from hospitalized SCD patients have been observed⁴¹, suggesting that C3 may indeed play an important role in the pathogenesis of VOC. The presence of inducible interactions between normal RBCs and adherent PMNs during inflammation suggests a yet undetermined physiological function for this phenomenon. It is possible that neutrophil captures may remove the damaged plasma membrane of older RBCs. Alternatively, the evidence of bidirectional signaling and the well established roles of RBCs in promoting platelet activation and thrombosis (e.g. through the release adenosine diphosphate (ADP)⁴² and other ADP-independent pathways⁴³), argue for a hemostatic role or a modulatory function in the inflammatory response. Numerous clinical studies have linked leukocytosis with ischemic heart disease⁴⁴. One can envision that the alterations of vascular permeability induced by leukocyte recruitment might be associated with a greater need for platelet adhesion to maintain vascular integrity during an inflammatory challenge. It is notable that RBC interactions were most prevalent at relatively low shear rates, which is consistent with a report where RBC adhesion to human PMNs in vitro were observed at shear rates <100 s⁻¹ using microcapillary flow chambers⁴⁵. PMN activation may cause vessel wall injury that could eventually lead to deep vein thrombosis⁴⁶.

Because G-CSF administration can enhance E-selectin ligand expression on human leukocytes⁴⁷, our data provide a mechanistic explanation for the severe complications observed in G-CSF-treated SCD patients, including the induction of sickle cell crises and acute lung injury^{16,48}, and the predisposition for TRALI in patients treated with this cytokine²⁴. Further, these observations offer novel candidate targets for the rational design of therapies for SCD, TRALI and possibly of other inflammatory or ischemic vascular diseases.

### Methods

Antibodies. Rat anti-mouse monoclonal antibodies anti-CD45 (30-F11), anti-Gr-1 (RB6-8C5), PE-anti-CD45 (30-F11), FITC-anti-LFA-1 (M174), APC- and PE-anti-L-selectin (MEL-14), APC-anti-Gr-1 (RB6-8C5), PE- and APC-anti-αM integrin (M1/70) and rat control isotype (IgG2b, κ) were obtained from BD Biosciences Pharmingen (San Diego, Calif.). Rat anti-mouse P-selectin (clone RB40.34), F4/80 and αM subunit (clone M1/70) antibodies were purified from supernatants of hybridoma cultures (ATCC, Manassas, Va.), and the anti-E-selectin (clone 9A9) was a gift of Dr. Barry Wolitzky (Immune Tolerance Network; Bethesda, Md.). The anti-MHC-I antibodies directed at the H2b (clone 28-8-6s; mouse IgG2a, κ) or H2d (clone 34-1-2s; mouse IgG2a, κ) haplotypes were purified from hybridoma supernatants (ATCC). Non-specific rat IgG and mouse IgG were obtained from Sigma-Aldrich (St. Louis, Mo.). AlexaFluor 488, 660, and 555 protein labeling kits were obtained from Invitrogen (Carlsbad, Calif.), and used to label anti-Gr-1, anti-F4/80, and anti-CD45, respectively, as per the manufacturer's instructions²⁶.

Mice. C57BL/6 and Balb/c mice were purchased from National Cancer Institute (Frederick, Md.). Berkeley sickle cell mice [Tg(Hu-miniLCRα1^{G}γ^{A}γδβ^{S}) Hbα^{-/-} Hbβ^{-/-}], referred to as SCD mice, and control hemizygous mice [Tg(Hu-miniLCRα1^{G}γ^{A}γδβ^{S}) Hbα^{-/-} Hbβ^{+/-}] mice have been previously described^{22,49}. Both SCD mice are from a mixed background (H2b haplotype with contributions from C57BL/6, 129Sv, FVB/N, DBA/2, Black Swiss)²². Cd44^{-/-}, Itgam^{-/-} and C3^{-/-} animals were purchased from the Jackson Laboratory (Bar Harbor, Me.). The Selplg^{-/-}, Selp^{-/-} and Sele^{-/-} were backcrossed into the C57BL/6 background for at least 7 generations. Genotypes of all mice were determined by PCR. All animals were housed at the Mount Sinai School of Medicine barrier facility. Experimental procedures performed on the animals were approved by the Animal Care and Use Committee of Mount Sinai.

Bone marrow transplantation. SCD mice with or without additional genetic deficiencies were generated by transplantation of bone marrow nucleated cells into lethally irradiated recipients as described²¹.

Generation of mice with knocked-down expression of ESL-1. Generation of lentiviral particles coding for the shRNA to knock-down the expression of ESL-1 or a control scrambled version was performed as published⁴. Donor lineage negative BM cells from wild-type donor mice were transduced with these lentiviral vectors and transplanted into lethally irradiated (1200 cGy, split doses 3 h apart) wild-type C57BL/6 recipients. Engraftment of recipient animals was assessed at least three weeks following transplantation by retroorbital bleeding and analysis of GFP+ leukocytes by flow cytometry.

Brightfield intravital microscopy. Brightfield intravital microscopy was performed as previously reported^{21,22}. For SCD mice, several postcapillary and collecting venules were recorded from 15 min after the surgical preparation (time=0 is surgical section of the cremaster) for 75 minutes (pre-TNF-α), with each venule recorded continuously for at least 2 min. SCD mice were then injected i.p. with 0.5 μg recombinant murine TNF-α (R & D Systems) and then the same venules were videotaped over a period of 90 min (91-180 min), after which venules were recorded again for another 90 min (181-270 min). The role of endothelial selectins was evaluated in identical experiments performed in Selp^{-/-} or Sele^{-/-} mice transplanted with BM cells from SCD donor mice, or using antibodies against P- or E-selectin (1 mg/kg) prior to surgery. In some experiments, 1 mg/kg of antibody against αM integrin (clone M1/70) subunit or IgG isotype control (IgG2b,κ) were infused through a carotid artery catheter into animals 70 min after treatment with TNF-α. In experiments with non-SCD C57BL/6 mice, TNF-α was administered intrascrotally and images recorded 160-210 min after cytokine injection. In some experiments, mice were injected with Src inhibitor PP2 (150 μg/kg), of SB203580 (100 μg; both from Calbiochem; San Diego, Calif.), or piceatannol (1 mg; Alexis Biochemicals; Lausen, Switzerland), or an equivalent volume of vehicle (dimethyl sulfoxide) 120 min after TNF-α administration.

Hemodynamic measurements and image analyses for brightfield intravital microscopy. The venular diameter was measured using a video caliper. Centerline RBC velocity was measured for each venule in real time using an optical Doppler velocimeter (Texas A&M, College Station, Tex.). Wall shear rate (γ) was calculated based on Poiseuille's law for a Newtonian fluid, γ=2.12 (8Vₘₑₐₙ) Dᵥ, where Dᵥ is the venule diameter, Vₘₑₐₙ is estimated as V_{RBC}/1.6, and 2.12 is a median empirical correction factor obtained from actual velocity profiles measured in microvessels in vivo. Blood flow rate was calculated from the formula: Vₘₑₐₙ×π×d²/4. All analyses were made using playback assessment of videotapes as previously described^{21,22}.

High-speed multichannel fluorescence intravital microscopy. Mice were prepared for intravital microscopy as indicated above. All movies were acquired with an Olympus BX61WI workstation using LumPlanF1 60× objective NA 0.90 W or 10× objective NA 0.30 W, as previously described²⁶ and analyzed using the SlideBook® software (Intelligent Imaging Innovations, Denver, Colo.).

Analysis of hemodynamic parameters in MFIM experiments. Centerline RBC velocities (V_{RBC}), wall shear rates (γ) and blood flow rates were calculated as indicated above. For experiments in which fluospheres were injected, V_{RBC} values were calculated by dividing the distance traveled by the fastest free flowing bead per frame by 0.022 s (capture rate: 45 frames per second=22 ms/frame).

In vivo analysis of αMβ2 activity. Yellow-green or red fluosphere® beads (1 μm in diameter, with excitation/emission of 505/515 nm and 580/605 nm, respectively; Molecular Probes, Eugene, Oreg.) were incubated with 1 mg/ml bovine serum albumin (Fisher Bioreagents; Fair Lawn, N.J.) for at least 2 h in phosphate-buffered saline and sonicated for 15 min in a water-bath sonicator (Laboratory Supplies Co., Hicksville, N.Y.) immediately before use. In some experiments, fluospheres were similarly coated with polyvinyl alcohol (MP Biomedicals; Solon, Ohio). Albumin-coated fluospheres (10⁹) were intravenously injected into mice prepared for intravital microscopy as indicated above, 180 min after intra-scrotal TNF-α administration. Images were captured 10 min after injection to allow clearance of fluosphere aggregates which appear in the first minutes. Image analyses were performed using the SlideBook® software.

Antibody-induced acute lung injury. Balb/c male mice (8-12 week-old) were injected i.v. with 3.5 mg/kg of anti-H2d (clone 34-1-2s) or control H2b (clone 28-8-6s) antibodies. After 2 h, BAL was performed on anesthetized mice by 3 washes with 1 ml PBS each using a 18G needle, and the protein content assessed by the bicinchoninic acid method (BCA; Pierce, Rockford, Ill.). In some experiments, mice were pre-treated as follows: rabbit anti-platelet serum (25 μl; i.p. injection; Accurate Chemicals; Westbury, N.Y.) diluted in 200 μl of PBS, 2 h prior anti-H2d treatment; 1 mg/kg i.v. injection of control or anti-P-selectin, E-selectin or anti-αMβ2 antibodies 5 min before anti-H2d; 150 mg/kg i.v. injection of n-acetyl cysteine 5 min before anti-H2d . Platelet and leukocyte counts from blood samples obtained at the end of the experiment were determined using a hematological Coulter® AC-T diff™ l Analyzer (Beckman Coulter, Miami, Fla.). None of these treatments affected circulating leukocyte counts (not shown). In the TRALI experiments involving intravital imaging of the cremasteric microcirculation, TNF-α was injected intrascrotally to allow the recruitment and imaging of inflammatory leukocytes.

Statistical analyses. Unless otherwise indicated, data are presented as mean±SEM. Parametric data were analyzed using the ANOVA t-test for two groups or with Tukey's multigroup comparison or Bonferroni's correction for more than two groups. Statistical significance for non-parametric distributions (RBC-leukocyte or fluosphere-leukocyte interactions, vascular permeability and ROS production) was assessed using the Mann-Whitney test for two groups or the Kruskal-Wallis test with Dunn's multigroup comparison for more than two groups. A p value below 0.05 was deemed significant. GraphPad Prism or Excel softwares were used for these analyses.

In vivo identification of adherent leukocytes. Mice were injected via a carotid artery catheter with AlexaFluor555 anti-CD45 (0.12 mg/kg), AlexaFluor488 anti-Gr-1 (0.12 mg/kg), and AlexaFluor660 anti-F4/80 (0.06 mg/kg) Abs, in 300 μL sterile PBS. Images in the FITC, Cy3 and Cy5 channels were recorded 0-90, 91-180 and 181-270 min after muscle exteriorization. Leukocyte identification was performed as indicated previously¹¹.

Analyisis of platelet interactions. Mice were injected with 1 μg of PE-conjugated anti-CD41 antibody to label intravascular platelets, and 0.02 mg/kg APC-conjugated anti-L-selectin antibody, and images captured as above in the Cy3, Cy5 and brightfield channels. CD41pos particles that remained attached to adherent leukocyte at least the duration of 1 frame (=2 s) were scored as leukocyte-platelet interactions.

Analysis of ESL-1 function. For the analysis of chimeric mice reconstituted with lentiviral-transduced BM cells, four to ten venules per mouse were analyzed 180 to 275 min after TNF-α treatment by acquisition of fluorescence (FITC channel for GFP) and brightfield images using 2×2 binning RBC-leukocyte interactions were analyzed from 1-2 min recordings and scored separately for those mediated by GFPneg and GFPpos leukocytes. The frequencies of interactions per leukocyte were averaged in each mouse and normalized relative to those found in the GFPneg group.

Analysis of leukocyte microdomains involved in RBC, platelet or fluosphere capture. For the RBC capture experiments, mice were injected with 0.02 mg/kg of a PE-conjugated anti-L-selectin antibody and images captured 180-250 min after TNF-μ treatment using the Cy3 and brightfield channels. For platelet capture experiments, mice were injected with 1 μg of PE-conjugated anti-CD41 antibody to label intravascular platelets, and 0.02 mg/kg APC-conjugated anti-L-selectin antibody, and images captured as above in the Cy3, Cy5 and brightfield channels. The sites of interaction were analyzed visually and scored as mediated by the “leading edge” if RBC or CD41pos platelets were found to interact with the area opposite to the L-selectin-enriched uropod which could be also distinguished by active formation of protruding lamellipodia and by the direction of leukocyte migration. 50% of the cell area on the side of the L-selectin cluster was scored as “trailing edge”. For the analysis of the microdomains involved in fluosphere capture, the same procedure was followed except that 109 albumin-coated fluospheres were injected when the L-selectin (using an APC-conjugated antibody) clusters were detectable, and images were acquired in the FITC, Cy5 and brightfield channels. The same criteria described above were used for microdomain identification.

Vascular permeability. To analyze anti-MHC-I-induced vascular damage, Balb/c mice were intravenously infused with 3.5 mg/kg of anti-MHC-I antibodies (to H2d or control H2b) and 1 mg of FITC-Dextran (70 Kd; Sigma-Aldrich) 210 min after TNF-α-treatment.

In some experiments, mice were pre-treated with 25 μL rabbit anti-platelet serum to deplete platelets, 1 mg/kg of control, anti-E-selectin or anti-αMβ2 antibodies, or 150 mg/kg of n-acetyl cysteine. Random fields containing small venules were imaged 30 min later using the FITC channel under a LumPlanFl 10× objective, NA 0.30 w. Images were masked over a threshold to quantify all FITC-Dextran-associated fluorescence intensity as well as the signal corresponding only to intravascular spaces. The percentage of extravasated FITC-Dextran was finally obtained using the formula: [(Total intensity−Intravascular intensity)/Total intensity]×100.

In vivo detection of leukocytes producing reactive oxygen species (ROS). Balb/c mice prepared for MFIM were injected i.v. with 29 nmoles of dihydroxyrhodamine-123 (DHR; Molecular Probes, Eugene, Oreg.). DHR uptaken by leukocytes actively producing ROS converts into fluorescent rhodamine-123 (507/529 nm) which can be detected by a bright punctuated pattern in the FITC channel (FIG. 11). In some experiments, mice were pre-treated with 25 μL rabbit anti-platelet serum to deplete platelets, 1 mg/kg of anti-E-selectin antibody, or 150 mg/kg of n-acetyl cysteine. Images were captured in the brightfield and FITC channels before and after injection of 3.5 mg/kg anti-H2d antibody, and the frequency of ROS-producing cells calculated out of all adherent leukocytes in each venular segment.

In vivo analysis of αMβ2 activity and expression. Images were captured for at least 30 s in the brightfield and FITC (for yellow-green fluospheres) channels. Adherent leukocytes were visually identified in the brightfield channel and the number of fluospheres associated to each leukocyte was counted. The average number of albumin-coated fluospheres bound to adherent intravascular leukocytes in a given 100 μm-long venular segment was used as a measure of αMβ2 activity, and was obtained from the formula: Fluospheres/WBC=total number of leukocyte-associated beads per venular segment/number of adherent leukocytes per venular segment. The number of fluospheres bound to individual leukocytes was used to obtain frequency histograms of fluosphere binding per adherent leukocyte in the different mouse groups (FIG. 5c-e and FIGS. 8 and 16).

For the analysis of chimeric mice reconstituted with lentiviral-transduced BM cells, 109 red fluospheres were intravenously injected 180 min after TNF-α treatment. After 10 min, 5 to 11 venules per mouse were analyzed by acquisition of fluorescence (FITC channel for GFP-expressing cells and Cy3 for red fluospheres) and brightfield images using 2×2 binning Beads bound to leukocytes were analyzed from 1-2 min recordings and scored separately for those mediated by GFPneg and GFPpos leukocytes. The average number of albumin-coated fluospheres bound to adherent intravascular leukocytes in a 100 μm-long venular segments was obtained from the formula: Beads/WBC=total number of leukocyte-associated beads in all venules analyzed in one mouse/number of adherent leukocytes in all venules analyzed in one mouse. The total number of bound fluospheres and the total leukocytes per leukocyte were averaged in each mouse and normalized relative to those found in the GFPneg group.

For the analysis of αMβ2 expression and fluosphere binding, mice were injected with 109 yellow-green fluospheres and 1 μg APC-conjugated anti-αM antibody 180 min after TNF-α administration. Images were captured in the brightfield, FITC (for fluospheres) and Cy5 (for anti-αM Ab) channels. Areas where adherent leukocytes were present, as determined from brightfield images, were analyzed for the mean intensity level in the Cy5 channel and the number of fluospheres present counted in the FITC channel. The mean intensity level in the Cy5 channel of an area similar to that of an adherent leukocyte was determined in leukocyte-free plasma for each vessel analyzed and subtracted from that of adherent leukocytes. The relative intensity associated to each cell was normalized to that of the leukocyte with the highest intensity of fluorescence for each mouse (100% expression).

Myeloperoxidase (MPO) activity assay to estimate PMN recruitment in lungs. Mice were sacrificed 2 h after anti-H2d antibody injection, the thoracic cavity exposed and a lung lobe excised and weighted. Lung samples were homogenized and sonicated in 1 mL of 0.05M potassium phosphate buffer, pH6.0 containing 0.5% hexadecyltrimethylammonium bromide (Sigma) at 4° C. and debris removed by centrifugation at 12,000 rpm for 10 min. The supernatant was collected and MPO activity in 10 μL of sample detected by adding 200 μL of tetramethylbenzidine substrate buffer (TMB, Sigma). After 2 min the reaction was stopped by addition of 50 μL of 1M HC1, and absorbance read at 450 nm in a μQuant ELISA plate reader (Biotek Instruments, Inc.; Winooski, Vt.). A standard curve using homogenates from known numbers of purified bone marrow PMNs was used to estimate the number of PMN in lungs.

Flow cytometry. Primary blood leukocytes were stained by incubation with 10 μg/mL of fluorescently-labeled antibodies to CD44, αM and L-selectin (all from BD Biosciences), or control antibody. Neutrophils were gated on the basis of low forward-scatter and high side-scatter. Samples were acquired using a FACSCalibur flow cytometer and analyzed using the Flowjo software (Treestar Inc., Ashland, Oreg.).

nRBC captures are mediated by the leading edge of adherent PMNs. Adherent leukocytes in venules of C57BL/6 mice treated with TNF-α were imaged following the intravenous injection of PE-conjugated anti-L-selectin (red, 0.5 μg) and FITC-conjugated anti-LFA-1 (clone M17/4; green, 1 μg). L-selectin clusters identify the trailing edge of adherent leukocytes. Brightfield images of nRBC interactions with PMNs in inflamed venules were captured 180 min after cytokine administration.

Platelets interact mostly with leukocyte microdomains at the leading edge. Platelets were labeled by anti-CD41 (red, 1 μg/mouse) and the trailing edge with anti-L-selectin (blue, 0.02 mg/Kg) in a TNF-□-stimulated mouse. Real time is shown in the left upper corner (h:min:s).

Regional activation of αMβ2 integrin at the leading edge of adherent leukocytes mediates fluosphere capture. TNF-α-treated C57BL/6 mice were injected with PE-conjugated anti-L-selectin (red; 0.5 μg) to label the trailing edge of adherent leukocytes. Fluosphere interactions with adherent leukocytes in inflamed venules were imaged 180 min after cytokine treatment, immediately upon the injection of albumin-coated fluospheres (green) through a catheter placed in the left carotid artery.

Platelet-WBC interactions are markedly induced by anti-H2d administration in Balb/c mice. Platelets were labeled by anti-CD41 (red, 1 μg/mouse) and the trailing edge with anti-L-selectin (blue, 0.02 mg/Kg). (a) Sequence of images just before anti-H2d administration. (b) Sequence of images taken after anti-H2d injection. Note the increase in platelet captures by leukocytes and also the interactions of non-labeled RBCs with adherent leukocytes.

### REFERENCES

1. BULLARD, D. C. ET AL. INFECTIOUS SUSCEPTIBILITY AND SEVERE DEFICIENCY OF LEUKOCYTE ROLLING AND RECRUITMENT IN E-SELECTIN AND P-SELECTIN DOUBLE MUTANT MICE. J EXP MED 183, 2329-36 (1996).

2. FRENETTE, P. S., MAYADAS, T. N., H., R., HYNES, R. O. & WAGNER, D. D. SUSCEPTIBILITY TO INFECTION AND ALTERED HEMATOPOIESIS IN MICE DEFICIENT IN BOTH P-AND E-SELECTINS. CELL 84, 563-574 (1996).

3. LABOW, M. A. ET AL. CHARACTERIZATION OF E-SELECTIN-DEFICIENT MICE: DEMONSTRATION OF OVERLAPPING FUNCTION OF THE ENDOTHELIAL SELECTINS. IMMUNITY 1, 709-720 (1994).

4. HIDALGO, A., PEIRED, A. J., WILD, M. K., VESTWEBER, D. & FRENETTE, P. S. COMPLETE IDENTIFICATION OF E-SELECTIN LIGANDS ON NEUTROPHILS REVEALS DISTINCT FUNCTIONS OF PSGL-1, ESL-1, AND CD44. IMMUNITY 26, 477-89 (2007).

5. ZARBOCK, A., LOWELL, C. A. & LEY, K. SPLEEN TYROSINE KINASE SYK IS NECESSARY FOR E-SELECTIN-INDUCED ALPHA(L)BETA(2) INTEGRIN-MEDIATED ROLLING ON INTERCELLULAR ADHESION MOLECULE-1. IMMUNITY 26, 773-83 (2007).

6. STEEGMAIER, M. ET AL. THE E-SELECTIN-LIGAND ESL-1 IS A VARIANT OF A RECEPTOR FOR FIBROBLAST GROWTH FACTOR. NATURE 373, 615-20 (1995).

7. LO, S. K. ET AL. ENDOTHELIAL-LEUKOCYTE ADHESION MOLECULE 1 STIMULATES THE ADHESIVE ACTIVITY OF LEUKOCYTE INTEGRIN CR3 (CD11B/CD18, MAC-1, ALPHA M BETA 2) ON HUMAN NEUTROPHILS. J EXP MED 173, 1493-500 (1991).

8. SIMON, S. I., HU, Y., VESTWEBER, D. & SMITH, C. W. NEUTROPHIL TETHERING ON E-SELECTIN ACTIVATES BETA 2 INTEGRIN BINDING TO ICAM-1 THROUGH A MITOGEN-ACTIVATED PROTEIN KINASE SIGNAL TRANSDUCTION PATHWAY. J IMMUNOL 164, 4348-58 (2000).

9. GAHMBERG, C. G., TOLVANEN, M. & KOTOVUORI, P. LEUKOCYTE ADHESION—STRUCTURE AND FUNCTION OF HUMAN LEUKOCYTE BETA2-INTEGRINS AND THEIR CELLULAR LIGANDS. EUR J BIOCHEM 245, 215-32 (1997).

10. BELLER, D. I., SPRINGER, T. A. & SCHREIBER, R. D. ANTI-MAC-1 SELECTIVELY INHIBITS THE MOUSE AND HUMAN TYPE THREE COMPLEMENT RECEPTOR. J EXP MED 156, 1000-9 (1982).

11. WANG, Y. ET AL. LEUKOCYTE ENGAGEMENT OF PLATELET GLYCOPROTEIN IBALPHA VIA THE INTEGRIN MAC-1 IS CRITICAL FOR THE BIOLOGICAL RESPONSE TO VASCULAR INJURY. CIRCULATION 112, 2993-3000 (2005).

12. BARREIRO, O., DE LA FUENTE, H., MITTELBRUNN, M. & SANCHEZ-MADRID, F. FUNCTIONAL INSIGHTS ON THE POLARIZED REDISTRIBUTION OF LEUKOCYTE INTEGRINS AND THEIR LIGANDS DURING LEUKOCYTE MIGRATION AND IMMUNE INTERACTIONS. IMMUNOL REV 218, 147-64 (2007).

13. NIETO, M. ET AL. POLARIZATION OF CHEMOKINE RECEPTORS TO THE LEADING EDGE DURING LYMPHOCYTE CHEMOTAXIS. J EXP MED 186, 153-8 (1997).

14. TOHYAMA, Y. ET AL. THE CRITICAL CYTOPLASMIC REGIONS OF THE ALPHAL/BETA2 INTEGRIN IN RAP1-INDUCED ADHESION AND MIGRATION. MOL BIOL CELL 14, 2570-82 (2003).

15. RIDLEY, A. J. ET AL. CELL MIGRATION: INTEGRATING SIGNALS FROM FRONT TO BACK. SCIENCE 302, 1704-9 (2003).

16. ABBOUD, M., LAVER, J. & BLAU, C. A. GRANULOCYTOSIS CAUSING SICKLE-CELL CRISIS. LANCET 351, 959 (1998).

17. HIRAHASHI, J. ET AL. MAC-1 SIGNALING VIA SRC-FAMILY AND SYK KINASES RESULTS IN ELASTASE-DEPENDENT THROMBOHEMORRHAGIC VASCULOPATHY. IMMUNITY 25, 271-83 (2006).

18. LOONEY, M. R., SU, X., VAN ZIFFLE, J. A., LOWELL, C. A. & MATTHAY, M. A. NEUTROPHILS AND THEIR FC GAMMA RECEPTORS ARE ESSENTIAL IN A MOUSE MODEL OF TRANSFUSION-RELATED ACUTE LUNG INJURY. J CLIN INVEST 116, 1615-23 (2006).

19. FRENETTE, P. S. & ATWEH, G. F. SICKLE CELL DISEASE: OLD DISCOVERIES, NEW CONCEPTS, AND FUTURE PROMISE. J CLIN INVEST 117, 850-8 (2007).

20. STUART, M. J. & NAGEL, R. L. SICKLE-CELL DISEASE. LANCET 364, 1343-60 (2004).

21. CHANG, J., SHI, P. A., CHIANG, E. Y. & FRENETTE, P. S. INTRAVENOUS IMMUNOGLOBULINS REVERSE ACUTE VASO-OCCLUSIVE CRISES IN SICKLE CELL MICE THROUGH RAPID INHIBITION OF NEUTROPHIL ADHESION. BLOOD 111, 915-23 (2008).

22. TURHAN, A., WEISS, L. A., MOHANDAS, N., COLLER, B. S. & FRENETTE, P. S. PRIMARY ROLE FOR ADHERENT LEUKOCYTES IN SICKLE CELL VASCULAR OCCLUSION: A NEW PARADIGM. PROC NATL ACAD SCI USA 99, 3047-51 (2002).

23. HOLNESS, L., KNIPPEN, M. A., SIMMONS, L. & LACHENBRUCH, P. A. FATALITIES CAUSED BY TRALI. TRANSFUS MED REV 18, 184-8 (2004).

24. SILLIMAN, C. C., AMBRUSO, D. R. & BOSHKOV, L. K. TRANSFUSION-RELATED ACUTE LUNG INJURY. BLOOD 105, 2266-73 (2005).

25. FALATI, S., GROSS, P., MERRILL-SKOLOFF, G., FURIE, B. C. & FURIE, B. REAL-TIME IN VIVO IMAGING OF PLATELETS, TISSUE FACTOR AND FIBRIN DURING ARTERIAL THROMBUS FORMATION IN THE MOUSE. NAT MED 8, 1175-81 (2002).

26. CHIANG, E. Y., HIDALGO, A., CHANG, J. & FRENETTE, P. S. IMAGING RECEPTOR MICRODOMAINS ON LEUKOCYTE SUBSETS IN LIVE MICE. NAT METHODS 4, 219-22 (2007).

27. TOTANI, L. ET AL. SRC-FAMILY KINASES MEDIATE AN OUTSIDE-IN SIGNAL NECESSARY FOR BETA2 INTEGRINS TO ACHIEVE FULL ACTIVATION AND SUSTAIN FIRM ADHESION OF POLYMORPHONUCLEAR LEUCOCYTES TETHERED ON E-SELECTIN. BIOCHEM J 396, 89-98 (2006).

28. SMITH, M. L., OLSON, T. S. & LEY, K. CXCR2- AND E-SELECTIN-INDUCED NEUTROPHIL ARREST DURING INFLAMMATION IN VIVO. J EXP MED 200, 935-9 (2004).

29. SIMON, S. I. ET AL. L-SELECTIN (CD62L) CROSS-LINKING SIGNALS NEUTROPHIL ADHESIVE FUNCTIONS VIA THE MAC-1 (CD11B/CD18) BETA 2-INTEGRIN. J IMMUNOL 155, 1502-14 (1995).

30. BUTCHER, E. C. LEUKOCYTE-ENDOTHELIAL CELL RECOGNITION: THREE (OR MORE) STEPS TO SPECIFICITY AND DIVERSITY. CELL 67, 1033-1036 (1991).

31. LAWRENCE, M. B. & SPRINGER, T. A. LEUKOCYTES ROLL ON A SELECTIN AT PHYSIOLOGIC FLOW RATES: DISTINCTION FROM AND PREREQUISITE FOR ADHESION THROUGH INTEGRINS. CELL 65, 859-873 (1991).

32. BARREIRO, O. ET AL. DYNAMIC INTERACTION OF VCAM-1 AND ICAM-1 WITH MOESIN AND EZRIN IN A NOVEL ENDOTHELIAL DOCKING STRUCTURE FOR ADHERENT LEUKOCYTES. J CELL BIOL 157, 1233-45 (2002).

33. CARMAN, C. V. & SPRINGER, T. A. A TRANSMIGRATORY CUP IN LEUKOCYTE DIAPEDESIS BOTH THROUGH INDIVIDUAL VASCULAR ENDOTHELIAL CELLS AND BETWEEN THEM. J CELL BIOL 167, 377-88 (2004).

34. LORANT, D. E. ET AL. INFLAMMATORY ROLES OF P-SELECTIN. J CLIN INVEST 92, 559-70 (1993).

35. EVANGELISTA, V. ET AL. PLATELET/POLYMORPHONUCLEAR LEUKOCYTE INTERACTION: P-SELECTIN TRIGGERS PROTEIN-TYROSINE PHOSPHORYLATION-DEPENDENT CD11B/CD18 ADHESION: ROLE OF PSGL-1 AS A SIGNALING MOLECULE. BLOOD 93, 876-85 (1999).

36. WANG, H. B. ET AL. P-SELECTIN PRIMES LEUKOCYTE INTEGRIN ACTIVATION DURING INFLAMMATION. NAT IMMUNOL 8, 882-92 (2007).

37. SHAPPELL, S. B. ET AL. MAC-1 (CD11B/CD18) MEDIATES ADHERENCE-DEPENDENT HYDROGEN PEROXIDE PRODUCTION BY HUMAN AND CANINE NEUTROPHILS. J IMMUNOL 144, 2702-11 (1990).

38. HUSEMANN, J., OBSTFELD, A., FEBBRAIO, M., KODAMA, T. & SILVERSTEIN, S. C. CD11B/CD18 MEDIATES PRODUCTION OF REACTIVE OXYGEN SPECIES BY MOUSE AND HUMAN MACROPHAGES ADHERENT TO MATRIXES CONTAINING OXIDIZED LDL. ARTERIOSCLER THROMB VASC BIOL 21, 1301-5 (2001).

39. ZARBOCK, A., SINGBARTL, K. & LEY, K. COMPLETE REVERSAL OF ACID-INDUCED ACUTE LUNG INJURY BY BLOCKING OF PLATELET-NEUTROPHIL AGGREGATION. J CLIN INVEST 116, 3211-9 (2006).

40. LUTZ, H. U. ET AL. NATURALLY OCCURRING ANTI-BAND-3 ANTIBODIES AND COMPLEMENT TOGETHER MEDIATE PHAGOCYTOSIS OF OXIDATIVELY STRESSED HUMAN ERYTHROCYTES. PROC NATL ACAD SCI USA 84, 7368-72 (1987).

41. WANG, R. H., PHILLIPS, G., JR., MEDOF, M. E. & MOLD, C. ACTIVATION OF THE ALTERNATIVE COMPLEMENT PATHWAY BY EXPOSURE OF PHOSPHATIDYLETHANOLAMINE AND PHOSPHATIDYLSERINE ON ERYTHROCYTES FROM SICKLE CELL DISEASE PATIENTS. J CLIN INVEST 92, 1326-35 (1993).

42. GAARDER, A., JONSEN, J., LALAND, S., HELLEM, A. & OWREN, P. A. ADENOSINE DIPHOSPHATE IN RED CELLS AS A FACTOR IN THE ADHESIVENESS OF HUMAN BLOOD PLATELETS. NATURE 192, 531-2 (1961).

43. SANTOS, M. T. ET AL. ENHANCEMENT OF PLATELET REACTIVITY AND MODULATION OF EICOSANOID PRODUCTION BY INTACT ERYTHROCYTES. A NEW APPROACH TO PLATELET ACTIVATION AND RECRUITMENT. J CLIN INVEST 87, 571-80 (1991).

44. COLLER, B. S. LEUKOCYTOSIS AND ISCHEMIC VASCULAR DISEASE MORBIDITY AND MORTALITY: IS IT TIME TO INTERVENE? ARTERIOSCLER THROMB VASC BIOL 25, 658-70 (2005).

45. GOEL, M. S. & DIAMOND, S. L. ADHESION OF NORMAL ERYTHROCYTES AT DEPRESSED VENOUS SHEAR RATES TO ACTIVATED NEUTROPHILS, ACTIVATED PLATELETS, AND FIBRIN POLYMERIZED FROM PLASMA. BLOOD 100, 3797-803 (2002).

46. WAKEFIELD, T. W., MYERS, D. D. & HENKE, P. K. MECHANISMS OF VENOUS THROMBOSIS AND RESOLUTION. ARTERIOSCLER THROMB VASC BIOL 28, 387-91 (2008).

47. DAGIA, N. M. ET AL. G-CSF INDUCES E-SELECTIN LIGAND EXPRESSION ON HUMAN MYELOID CELLS. NAT MED 12, 1185-90 (2006).

48. ARIMURA, K. ET AL. ACUTE LUNG INJURY IN A HEALTHY DONOR DURING MOBILIZATION OF PERIPHERAL BLOOD STEM CELLS USING GRANULOCYTE-COLONY STIMULATING FACTOR ALONE. HAEMATOLOGICA 90, ECR10 (2005).

49. PASZTY, C. ET AL. TRANSGENIC KNOCKOUT MICE WITH EXCLUSIVELY HUMAN SICKLE HEMOGLOBIN AND SICKLE CELL DISEASE. SCIENCE 278, 876-8 (1997).

Various publications are cited herein, the contents of which are hereby incorporated by reference in their entireties.

What is claimed is:

1. A method for reducing inflammation in a subject in need of such treatment comprising administering to the subject one or more compound in an amount effective to (a) inhibit activity of E-selectin, (b) inhibit activity of an Src family member, or (c) inhibit activity of Mac-1.

2. The method of claim 1, wherein the Src family member is selected from the group consisting of Src, Yes, Fyn, Fgr, Lck, Hck, Blk, Lyn and Frk.

3. The method of claim 1, wherein the one or more compound is an antibody selected from the group consisting of an E-selectin antibody, a Mac-1 antibody, an ESL-1 antibody, and an antibody to an Src family member.

4. The method of claim 1, wherein the one or more compound is a molecule selected from the group consisting of an antisense molecule and an RNAi molecule.

5. The method of claim 4, wherein the compound inhibits the expression of a gene selected from the group consisting of E-selectin, ESL-1, CD11b, CD18 or an Src family member.

6. The method of claim 1, wherein the compound is selected from the group consisting of A205804, CGP76030, INNO-406, SU6656, PP1, PP2 and Dasatinib

7. The method of claim 1, further comprising administering to the subject one or more compound in an amount effective to reduce the activity of a neutrophil generated reactive oxygen species.

8. The method of claim 7, wherein the compound is selected from the group consisting of an enzyme, an antioxidant, and a reactive oxygen species scavenger.

9. The method of claim 8, wherein the enzyme is selected from the group consisting of a superoxide dismutase, catalase, peroxidase and a peroxiredoxin.

10. The method of claim 8, wherein the antioxidant is selected from the group consisting of ascorbic acid, tocopherol, uric acid, and glutathione.

11. The method of claim 8, wherein the reactive oxygen species scavenger is selected from the group consisting of n-acetyl-cysteine, DPI, diphenyleneiodonium chloride and polyphenol.

12. The method of claim 1, wherein the compound reduces binding of a neutrophil to a platelet.

13. The method of claim 1, wherein the subject is a human.

14. The method of claim 1, wherein the tissue is lung tissue.

15. The method of claim 1, wherein inflammation-associated tissue damage is reduced in the subject.

16. A method of treating an inflammation-associated disorder in a subject comprising administering to the subject one or more compound in an amount effective to (a) inhibit the activity of E-selectin, (b) inhibit activity of an Src family member, or (c) inhibit the activity of Mac-1.

17. The method of claim 16, wherein the disorder is selected from the group consisting of Transfusion-Related Acute Lung Injury and vaso-occlusion.
